(19) United States
(12) Patent Application Publication
(10) Pub. No.: US 2008/0058656 A1
(43) Pub. Date: Mar. 6, 2008

(54) ELECTRIC TOMOGRAPHY

(76) Inventors: Benedict J. Costello, Berkeley, CA (US); Timothy Robertson, Belmont, CA (US); Mark J. Zdeblick, Portola Valley, CA (US); George M. Savage, Portola Valley, CA (US); Olivier Colliou, Los Gatos, CA (US); Todd Thompson, San Jose, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(21) Appl. No.: 11/731,786

(22) Filed: Mar. 30, 2007

Related U.S. Application Data
(63) Continuation-in-part of application No. PCT/US05/36035, filed on Oct. 6, 2005.
(63) Continuation-in-part of application No. PCT/US06/12246, filed on Mar. 31, 2006.
(60) Provisional application No. 60/617,618, filed on Oct. 8, 2004.
(60) Provisional application No. 60/665,145, filed on Mar. 25, 2005.
(60) Provisional application No. 60/696,321, filed on Jun. 30, 2005.
(60) Provisional application No. 60/705,900, filed on Aug. 5, 2005.
(60) Provisional application No. 60/667,575, filed on Mar. 31, 2005.
(60) Provisional application No. 60/667,529, filed on Mar. 31, 2005.
(60) Provisional application No. 60/684,751, filed on May 25, 2005.
(60) Provisional application No. 60/695,577, filed on Jun. 29, 2005.
(60) Provisional application No. 60/790,507, filed on Apr. 7, 2006.
(60) Provisional application No. 60/797,403, filed on May 2, 2006.

(51) Int. Cl. A61B 5/02

(52) U.S. Cl. 600/508; 600/595; 607/9

(57) ABSTRACT

Methods for evaluating motion of a tissue, such as of a cardiac location, e.g., heart wall, via electrical field tomography are provided. In the subject methods, an sensing element is stably associated with a tissue location of interest. Signals obtained from the sensing element are obtained to evaluate movement of the tissue location. Also provided are systems and devices for practicing the subject methods. In addition, innovative data displays and systems for producing the same are provided. The subject methods and devices find use in a variety of different applications, including cardiac resynchronization therapy.

### CROSS-REFERENCE TO RELATED APPLICATIONS

This application is a continuation-in-part application of U.S. application Ser. No. PCT/US2005/036035 filed on Oct. 6, 2005; which application pursuant to 35 U.S.C. § 119 (e) claims priority to the filing date of: U.S. Provisional Patent Application Ser. No. 60/617,618 filed Oct. 8, 2004; U.S. Provisional Patent Application Ser. No. 60/665,145 filed Mar. 25, 2005; U.S. Provisional Patent Application Ser. No. 60/696,321 filed Jun. 30, 2005; and U.S. Provisional Patent Application Ser. No. 60/705,900 filed Aug. 5, 2005; the disclosures of which applications are all herein incorporated by reference.

This application is also a continuation-in-part application of U.S. application Ser. No. PCT/US2006/012246 filed on Mar. 31, 2006; which application pursuant to 35 U.S.C. § 119 (e) claims priority to the filing date of: U.S. Provisional Patent Application Ser. No. 60/667,575 filed Mar. 31, 2005 United States Provisional Patent Application Serial No. 60/667,529 filed Mar. 31, 2005; U.S. Provisional Patent Application Ser. No. 60/684,751 filed May 25, 2005; and U.S. Provisional Patent Application Ser. No. 60/695,577 filed Jun. 29, 2005; the disclosures of which applications are all herein incorporated by reference.

Pursuant to 35 U.S.C. § 119 (e), this application also claims priority to the filing dates of U.S. Provisional Application Ser. Nos. 60/790,507 titled “Tetrahedral Electrode Tomography,” and filed on Apr. 7, 2006 and 60/797,403 titled “Continuous Field Tomography,” and filed on May 2, 2006; the disclosures of which are herein incorporated by reference.

### INTRODUCTION

In a diverse array of applications, the evaluation of tissue motion is desirable, e.g., for diagnostic or therapeutic purposes. An example of where evaluation of tissue motion is desirable is cardiac resynchronization therapy (CRT), where evaluation of cardiac tissue motion as observed by traditional ultrasound techniques is employed for diagnostic and therapeutic purposes.

CRT is an important new medical intervention for patients suffering from heart failure, e.g., congestive heart failure (CHF). When congestive heart failure occurs, symptoms develop due to the heart's inability to function sufficiently. Congestive heart failure is characterized by gradual decline in cardiac function punctuated by severe exacerbations leading eventually to death. It is estimated that over five million patients in the United States suffer from this malady.

The aim of resynchronization pacing is to induce the interventricular septum and the left ventricular free wall to contract at approximately the same time. Resynchronization therapy seeks to provide a contraction time sequence that will most effectively produce maximal cardiac output with minimal total energy expenditure by the heart. The optimal timing is calculated by reference to hemodynamic parameters such as dP/dt, the first time-derivative of the pressure waveform in the left ventricle. The dP/dt parameter is a well-documented proxy for left ventricular contractility.

In current practice, external ultrasound measurements are used to calculate dP/dt. Such external ultrasound is used to observe wall motion directly. Most commonly, the ultrasound operator uses the ultrasound system in a tissue Doppler mode, a feature known as Tissue Doppler Imaging (TDI), to evaluate the time course of displacement of the septum relative to the left ventricle free wall. The current view of clinicians is that ultrasonographic evaluation using TDI or a similar approach may become an important part of qualifying patients for CRT therapy.

As currently delivered, CRT therapy is effective in about half to two-thirds of patients implanted with a resynchronization device. In approximately one-third of these patients, this therapy provides a two-class improvement in patient symptoms as measured by the New York Heart Association scale. In about one-third of these patients, a one-class improvement in cardiovascular symptoms is accomplished. In the remaining third of patients, there is no improvement or, in a small minority, a deterioration in cardiac performance. This group of patients is referred to as non-responders. It is possible that the one-class New York Heart Association responders are actually marginal or partial responders to the therapy, given the dramatic results seen in a minority.

The synchronization therapy, in order to be optimal, targets the cardiac wall segment point of maximal delay, and advances the timing to synchronize contraction with an earlier contracting region of the heart, typically the septum. However, the current placement technique for CRT devices is usually empiric. A physician will cannulate a vein that appears to be in the region described by the literature as most effective. The device is then positioned, stimulation is carried out, and the lack of extra-cardiac stimulation, such as diaphragmatic pacing, is confirmed. With the currently available techniques, rarely is there time or means for optimizing cardiac performance.

When attempted today, clinical CRT optimization must be performed by a laborious manual method of an ultrasonographer evaluating cardiac wall motion at different lead positions and different interventricular delay (IVD) settings. The IVD is the ability of pacemakers to be set up with different timing on the pacing pulse that goes to the right ventricle versus the left ventricle. In addition, all pacemakers have the ability to vary the atrio-ventricular delay, which is the delay between stimulation of the atria and the ventricle or ventricles themselves. These settings can be important in addition to the location of the left ventricular stimulating electrode itself in resynchronizing the patient.

Current use of Doppler to localize elements in the heart have been limited to wall position determination via external ultrasonography, typically for purposes of measuring valve function, cardiac output, or rarely, synchronization index.

### SUMMARY

Methods for evaluating tissue motion, such as of a cardiac tissue motion, e.g., heart wall motion, via electric tomography are provided. In the subject methods, an electric field sensing element is stably associated with a site of the tissue of interest, and a property of, e.g., a change in, the electric field sensed by the sensing element is employed to evaluate movement of the tissue. Also provided are devices and systems for practicing the subject methods. In certain embodiments, innovative data processing and display protocols, as well as systems that provided for the same, are provided. The subject methods, devices and systems find use in a variety of different applications, such as cardiac related applications, e.g., cardiac resynchronization therapy, and other applications.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIGS. 1 to 2G provide depictions of various electrical tomography system embodiments of the subject invention.

FIGS. 3A to 5 provide a view of an electrode configuration that finds use in electrical gradient tomography applications of the present invention, as well explanatory graphs and electric field maps therefore.

FIG. 6 provides a view of two electrode rings used in tandem for a tetrahedral configuration.

FIG. 7 provides a view of a three electrode ring used with a solid ring electrode for a tetrahedral configuration.

FIG. 8 provides a view of a quadrant electrode configured to allow for a tetrahedral configuration.

FIG. 9 provides a view of a system according to a representative embodiment of the invention.

FIG. 10 illustrates an exemplary configuration for electrical tomography, in accordance with an embodiment of the present invention.

FIG. 11 illustrates an exemplary configuration for 3-D electrical tomography, in accordance with an embodiment of the present invention.

FIG. 12 illustrates an electrical tomography system based on an existing pacing system, in accordance with an embodiment of the present invention.

FIG. 13 illustrates a schematic circuit diagram for the voltage-driving and data-acquisition system 1904 in FIG. 12, in accordance with an embodiment of the present invention.

FIG. 14 illustrates a configuration for driving electrodes to mitigate effects caused by large electrode interface impedance in an electrical tomography system, in accordance with an embodiment of the present invention.

FIG. 15 illustrates a schematic circuit diagram showing an exemplary implementation of a frequency-division-multiplexing system for simultaneously transmitting multiple electrical tomography signals over a single wire, in accordance with an embodiment of the present invention FIG. 16 illustrates data showing high correlation of electrical tomography data and Tissue Doppler Imaging data.

FIG. 17 provides a three-dimensional ET motion display of two left ventricular electrodes with a corresponding EKG according to a representative embodiment of the invention.

FIG. 18 provides another embodiment of a three-dimensional ET motion display of a left ventricular electrode displayed with a corresponding EKG, respiration data, and velocity plot according to a representative embodiment of the invention.

FIG. 19 provides a graphical user interface (GUI) according to a representative embodiment of the invention.

FIG. 20 provides a graphical user interface (GUI) of cardiac performance parameters both at baseline and during pacing according to a representative embodiment of the invention.

FIG. 21 provides a graphical user interface (GUI) of cardiac performance parameters over time according to a representative embodiment of the invention.

FIG. 22 provides an embodiment of a method for evaluating a three-dimensional volume bounded by four or more electrodes, according to a representative embodiment of the invention.

FIG. 23 illustrates a schematic diagram showing an exemplary implementation of methods of data acquisition, data processing, and display, in accordance with an embodiment of the invention.

### DETAILED DESCRIPTION

Methods for evaluating motion of a tissue, such as of a cardiac tissue, e.g., a heart wall, via electric tomography are provided. In embodiments of the methods, an electric field sensing element is stably associated with a site of the tissue of interest, and a property of the electric field, e.g., a change in the electric field perceived by the sensing element, is employed to evaluate movement of the tissue. Also provided are systems devices for practicing the subject methods. In addition, also disclosed are innovative data processing and display protocols, and systems for performing the same. The subject methods and devices find use in a variety of different applications, e.g., cardiac resynchronization therapy.

In further describing the subject invention, aspects of electrical field tomography methods are reviewed first in greater detail. Next, embodiments of electric field tomography devices and systems are described in greater detail, both generally and in terms of specific embodiments of devices and systems that may be employed in such embodiments. Next, embodiments of innovative data processing and display protocols and systems for practicing the same are reviewed. Following this section, embodiments of applications in which the subject invention finds use are described, as well as other aspects of the invention, such as computer related embodiments and kits that find use in practicing the invention.

Electric Tomography Methods

As summarized above, the subject invention provides electric tomography methods for evaluating movement of a tissue location of interest. In the subject tomography methods, data obtained by a sensing element stably associated with the tissue location of interest as it moves through an applied electric field are employed. While the methods may be viewed as tomography methods, such a characterization does not mean that the methods are necessarily employed to obtain a map of a given tissue location, such as a 2-dimensional or 3-dimensional map, but instead just that changes in a sensing element as it moves through an applied electric field are used to evaluate or characterize a tissue location in some way. However, in certain embodiments the data obtained may be processed to obtain and display virtual represent

By “electric field tomography method” is meant a method which employs detected changes in an applied electric field to obtain a signal, which signal is then employed to determine tissue location movement. For the purposes of this application, the term “electric field” means an electric field from which tomography measurement data is obtained. The electric field is one or more cycles of a sine wave. There is no necessary requirement for discontinuity in the field to obtain data. As such, the applied field employed in embodiments of the subject invention is continuous over a given period of time.

The “electric field” used for tomography measurement may, at times, be provided with disruptions or naturally have some disruptions, and still be considered a “continuous field”. As clarifying examples, pulsing the field to conserve power or mutiplexing between different fields remains within the meaning of “continuous field” for the purposes of the present invention. In contrast, a time-of-flight detection method falls outside of the meaning of “continuous field” for the purposes of the present invention. Accordingly, the continuous field applied in the subject methods is distinguished from “time of flight” applications, in which a duration-limited signal or series of such signals is emitted from a first location and the time required to detect the emitted signal at a second location is employed to obtain desired data. At best, if a series of signals are generated in a time of flight application, the series of signals is discontinuous, and therefore not a continuous field, such as the field employed in the present invention.

The underlying precept among the electric field tomography method is that a source is provided which generates a field ψ. ψ varies throughout the internal anatomical area of interest.

One example of the source field ψ can be expressed in a form:
ψ=A sin (2πf t+φ)
where:

f is the frequency,

φ is a phase,

A is the amplitude, and

t is time.

In certain embodiments, the field oscillates as a function of time, and can be described simply an AC field.

In obtaining data from the electric field, A, f or φ is a function of some parameter(s) of interest. Two parameters of interest among the many available parameters are location position and location velocity. When one or more properties of the field, e.g., A, f and/or φ, is sampled at various points, and the measured property is compared to the reference value, electrical tomography data is obtained.

For example, if an electrical field driven by an alternating-current (AC) voltage is present in a tissue region, one may detect an induced voltage on an electrode therein. The frequency of the induced voltage, f′, is the same as the frequency of the electrical field. The amplitude of the induced signal, however, varies with the location of the electrode. Hence, by detecting the induced voltage and by measuring the amplitude of the signal, one can determine the location as well as the velocity of the electrode.

In general, electric field tomography can be based upon measurement of the amplitude, frequency, and phase shift of the induced signal. Further details regarding the underlying operating principles of electrical field tomography are provided in PCT application serial no. PCT/US2005/036035; the disclosure of which is herein incorporated by reference.

As summarized above, the subject invention provides methods of evaluating movement of a tissue location. “Evaluating” is used herein to refer to any type of detecting, assessing or analyzing, and may be qualitative or quantitative. In representative embodiments, movement is determined relative to another tissue location, such that the methods are employed to determine movement of two or more tissue locations relative to each other.

The tissue location(s) or site(s) is generally a defined location (i.e. site) or portion of a body, i.e., subject, where in many embodiments it is a defined location or portion (i.e., domain or region) of a body structure, such as an organ, where in representative embodiments the body structure is an internal body structure, such as an internal organ, e.g., heart, kidney, stomach, lung, etc. In representative embodiments, the tissue location is a cardiac location. As such and for ease of further description, the various aspects of the invention are now reviewed in terms of evaluating motion of a cardiac location. The cardiac location may be either endocardial or epicardial, as desired, and may be an atrial or ventricular location. Where the tissue location is a cardiac location, in certain embodiments, the cardiac location is a heart wall location, e.g., a chamber wall, such as a ventricular wall, a septal wall, etc. Although the invention is now further described in terms of cardiac motion evaluation embodiments, the invention is not so limited, the invention being readily adaptable to evaluation of movement of a wide variety of different tissue locations.

In practicing embodiments of the invention, following implantation of any required elements in a subject (e.g., using known surgical techniques), the first step is to set up or produce, i.e., generate, an electric field in a manner such that the tissue location(s) of interest is present in the generated electric field. In certain embodiments, a single electric field is generated, while in other embodiments a plurality of different electric fields are generated, e.g., two or more, such as three or more, e.g., four or more, six or more, etc., where in certain of these embodiments, the generated electric fields may be substantially orthogonal to one another. Of interest in certain embodiments are multiple electrical fields as described in U.S. patent application Ser. No. 11/562,690 filed Nov. 22, 2006 and PCT application serial no. PCT/ US06/61223 filed Nov. 22, 2006; the disclosures of which are herein incorporated by reference.

An electric field can be generated such that the voltages applied to two or more electrodes can be adjusted to synthesize a “virtual electrode,” such that the effective position to which the electric fields return is not coincident with either electrode. For example, if three electrodes are positioned at the vertices of an equilateral triangle, and one of the electrodes is selected as ground, while the other two electrodes are energized at the same voltage, the effective direction of the field will be from the ground electrode to a point halfway between the two positive electrodes. By varying the relative voltages on the positive electrodes, the direction of the field can be “steered” to a direction that falls between the two electrodes. By moving the ground electrode, or by varying the voltage on one, two, or all three electrodes, for example, the direction of an electric field can be “steered” or oriented in any arbitrary direction, e.g. in a direction of motion of interest. In certain embodiments, the electric field(s) can be reoriented at least once over a given period of time. The capacity to change orientation of the electric fields and create distinct electrical fields in each of multiple planes can improve resolution in characterizing intracardiac wall motion.

The precision of the “steering”, or the ability to select the direction of the electric field, can be increased by adding more electrodes (e.g. around a ring external to the body, or on a lead). In one embodiment, a belt with many segmented electrodes can be placed around the chest of a subject. By choosing the appropriate linear combination of voltages on the segments, a relatively flat electric field can be generated in an arbitrary orientation. Several fields of different frequency can be superimposed in the same configuration. In certain embodiments, a single electric field is generated, and in some embodiments, two fields that are substantially orthogonal over a large area can be generated. In certain embodiments a plurality of different electric fields can be generated, e.g., two or more, such as three or more, e.g., four or more, six or more, etc., where in certain of these embodiments, the generated electric fields may be substantially orthogonal to one another. In certain embodiments, electric field are generated as described in U.S. application Ser. No. 11/562,690 titled “External Continuous Field Tomography,” filed Nov. 22, 2006; the disclosure of which is herein incorporated by reference.

In practicing the subject methods, the applied electric field(s) may be applied using any convenient format, e.g., from outside the body, from an internal body site, or a combination thereof, as long as the tissue location(s) of interest resides in the applied electric field. The electric field or fields employed in the subject methods may be produced using any convenient electric field generation element, where in certain embodiments the electric field is set up between a driving electrode and a ground element, e.g., a second electrode, an implanted medical device that can serve as a ground, such as a “can” of an implantable cardiac device (e.g., pacemaker), etc. The electric field generation elements may be implantable such that they generate the electric field from within the body, or the elements may be ones that generate the electric field from locations outside of the body, or a combination thereof. As such, in certain embodiments the applied electric field is applied from an external body location, e.g., from a body surface location. In yet other embodiments, the electric field is generated from an internal site, e.g., from an implanted device (e.g. a pacemaker can), one or more electrodes on a lead, such as a multiplexed electric lead (e.g., as described in U.S. patent application Ser. No. 10/734490; the disclosure of which is herein incorporated by reference); including a segmented electrode lead (e.g., as described in PCT Patent Application Serial No. PCT/US2006/ 48944; the disclosure of which is herein incorporated by reference).

In certain embodiments, the electric field is a radiofrequency or RF field. As such, in these-embodiments, the electric field generation element generates an alternating current electric field, e.g., that comprises an RF field, where the RF field has a frequency ranging from about 1 kHz to about 100 GHz or more, such as from about 10 kHz to about 10 MHz, including from about 25 KHz to about 1 MHz. Aspects of this embodiment of the present invention involve the application of alternating current within the body transmitted between two electrodes with an additional electrode pair being used to record changes in a property, e.g., amplitude, within the applied RF field. Several different frequencies can be used to establish different axes and improve resolution, e.g., by employing either RF energy transmitted from a subcutaneous or cutaneous location, in various planes, or by electrodes, deployed for example on an inter-cardiac lead, which may be simultaneously used for pacing and sensing. Where different frequencies are employed simultaneously, the magnitude of the difference in frequencies will, in certain embodiments, range from about 100 Hz to about 100 KHz, such as from about 5 KHz to about 50 KHz. Amplitude information can be used to derive the position of various sensors relative to the emitters of the alternating current.

In embodiments of the methods, following generation of the applied electric field, as described above, a signal (representing data) from an electric field sensing element that is stably associated with the target tissue location of interest is then detected to evaluate movement of the tissue location. In certain embodiments, a signal from the sensing element is detected at least twice over a duration of time, e.g., to determine whether a parameter(s) being sensed by the sensing element has changed or not over the period of time, and therefore whether or not the tissue location of interest has moved over the period of time of interest. In certain embodiments, a change in a parameter is detected by the sensing element to evaluate movement of the tissue location. In certain embodiments, the detected change may also be referred to as a detected “transformation,” as defined above. Parameters of interest include, but are not limited to: amplitude, phase and frequency of the applied electric field, as reviewed in greater detail below. In certain embodiments, the parameter of interest is detected at the two or more different times in a manner such that one or more of the other of the three parameters is substantially constant, if not constant. In a given embodiment, the sensing element can provide output in an interval fashion or continuous fashion for a given duration of time, as desired.

By “stably associated with” is meant that the sensing element is substantially if not completely fixed relative to the tissue location of interest, such that when the tissue location of interest moves, the sensing element also moves. As the employed electric field sensing element is stably associated with the tissue location, its movement is at least a proxy for, and in certain embodiments is the same as, the movement of the tissue location to which it is stably associated, such that movement of the sensing element can be used to evaluate movement of the tissue location of interest. The electric field sensing element may be stably associated with the tissue location using any convenient approach, such as by attaching the sensing element to the tissue location by using an attachment element, such as a hook, etc.; by having the sensing element on a structure that compresses the sensing element against the tissue location or is temporarily fixed in position (e.g. a sensing element on a lead or guidewire) such that the two are stably associated; etc. The sensing element may be on a standalone implanted device, or on a carrier, e.g., a lead, guidewire, sheath, etc.

In certain embodiments, a single sensing element is employed. In such methods, evaluation may include monitoring movement of the tissue location over a given period of time. Such embodiments may further include instances where two or more different locations are monitored sequentially, such that a first location is monitored and then the sensing element is moved to a second location which is monitored. For example, a single sensing element may be used to monitor a first location (e.g. an electrode on a cardiac lead at a first location in a cardiac vein) and then the sensing element is moved to a second location which is monitored (e.g. the electrode is placed at a second location in a cardiac vein).

In certain embodiments, two or more distinct sensing elements are employed to evaluate movement of two or more distinct tissue locations. The number of different sensing elements that are employed in a given embodiment may vary greatly, where in certain embodiments the number employed is 2 or more, such as 3 or more, 4 or more, 5 or more, 8 or more, 10 or more, etc. In such multi-sensor embodiments, the methods may include evaluating movement of the two or more distinct locations relative to each other.

The sensing element is, in certain embodiments, an electric potential sensing element, such as an electrode. In these embodiments, the sensing element provides a value for a sensed electric potential which is a function of the location of the sensing element in the generated electric field. In certain embodiments, the electric field sensing element is an electrode. The electrode may be present as a stand alone device, e.g., a small device that wirelessly communicates with a data receiver, or part of a component device, e.g., a medical carrier, such as a lead. Where the sensing element is an electrode on a lead, the lead may be a conventional lead that includes a single electrode. In alternative embodiments, the lead may be a multi-electrode lead that includes two or more different electrodes, where in certain of these embodiments, the lead may be a multiplex lead that has two or more individually addressable electrodes electrically coupled to the same wire or wires. In certain embodiments, a lead, such as a cardiovascular lead, is employed that includes one or more sets of electrode satellites (e.g., that are electrically coupled to at least one elongated conductive member, e.g., an elongated conductive member present in the lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; the disclosures of which applications are herein incorporated by reference.

In certain embodiments, the multiplex lead includes satellite electrodes that are segmented electrodes, in which two or more different individually addressable electrodes are couple to the same satellite controller, e.g., integrated circuit, present on the lead. Segmented electrode structures of interest include, but are not limited to, those described in: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various semented multiplex lead structures of these applications being herein incorporated by reference.

In certain embodiments, the subject methods include providing a system that includes: (a) an electric field generation element; and (b) an electric field sensing element that is stably associated with the tissue location of interest. This providing step may include either implanting one or more new elements into a body, or simply employing an already existing implanted system, e.g., a pacing system, for example by using an adapter (for example a module that, when operationally connected to a pre-existing implant, enables the implant to perform the subject methods), as described below. This step, if employed, may be carried out using any convenient protocol.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

The subject methods result in the generation of data in the form of signals, where the signals are tissue movement dependent. From changes determined in these signals obtained from the electric field sensing element, the dynamics and timing of tissue movement can be derived. This rich source of data allows the generation of both physical anatomical dimensions and the physiological functions which they bespeak, typically in real time.

The tissue movement evaluation data obtained using the subject methods may be employed in raw or processed format, as desired and depending on the particular application. In certain embodiments, the obtained data may be processed and displayed to a user, e.g., in the form a computer display, as a graphical user interface (GUI), etc.

The tissue movement evaluation data obtained using the subject methods may be employed in a variety of different applications, including but not limited to, monitoring applications, treatment applications, etc. Applications in which the data obtained from the subject methods finds use are further reviewed in greater detail below.

In certain embodiments, the methods and systems only determine the relative timing and distance along the line of position of, for example, two electrodes, one with respect to another. By using multiple frequencies and multiple electrode pairs, multiple lines of position can be derived, improving the resolution of this system with respect to determining inter- or and/or intra-ventricular synchrony of a given heart.

Electrical Gradient Tomography

In certain embodiments, electrical gradient tomography is employed. Using electrical gradient tomography, the precise location of the electrodes in the subject can be estimated. This estimation of position is accomplished by determining the rate of change of the AC signal as a function of distance in more than one direction. This rate of change is a function of distance as the gradient of the AC potential.

The electrical gradient tomography embodiment of the invention measures the AC potential at a location between two different electrodes. AC voltage is employed at both the drive electrode and the receive electrode. The receive electrode is placed in a different position in the body from the drive electrode. In the simplest form of the current tomography invention, the variation in amplitude at the receive electrode is related to the distance between the ground electrode and the drive electrode.

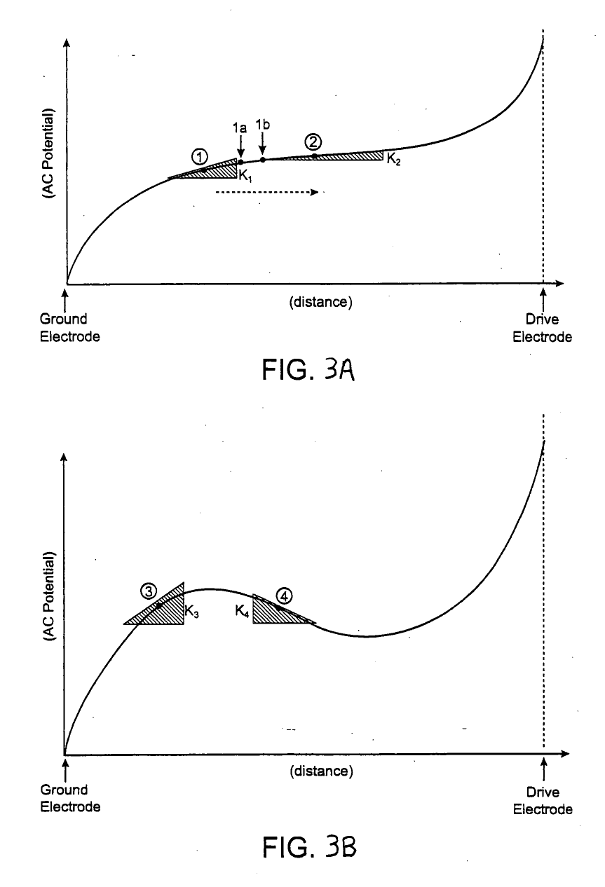

By measuring the gradient of the AC potential, as well as the AC potential at the receive electrode location, both the absolute value and the rate of change of the value is achieved. From the voltage data a position signal can be calculated for an object or location (e.g. an electrode, or a tissue location), and by evaluating the rate of change of the position signal, the position as a function of time can be determined. The velocity can be computed by differentiating, or taking the derivative of, the position signal of the object (e.g. an electrode). The velocity of an object (e.g. an electrode, or a tissue location) is its speed in a particular direction, or the rate of displacement, and indicates both the speed and direction of an object. In some embodiments, the velocity is computed in a linear direction. In some embodiments, the computed velocity is a linear velocity computed in the direction of maximum motion. From this information, more accurate data of the motion of that receive electrode as a function of time is accomplished. FIGS. 3, 4, 5, and 6 illustrate various aspects of electrical gradient tomography embodiments of the methods.

FIG. 3A provides an example of a relatively smoothly operating system among those of the present invention. The AC potential of the receive electrode is plotted as a function of the distance between the ground electrode and receive electrode. From left to right, this plot is a monotonic, smooth function. However, the plot is not linear. The plot is grossly nonlinear near the electrodes, i.e., near the drive electrode and near the ground electrode.

FIG. 3B provides an example of data which can be improved using electrical gradient tomography. As with the prior example, the data to be improved is the potential of the receive electrode as a function of distance between ground electrode and the drive electrode. In this case, however, the potential drops at closer distances to one electrode.

There is an unusual way of analyzing this phenomenon which leads to some of the special advantages of electrical gradient tomography. There are two situations involved. One is where the drive electrode is moving relative to the ground electrode. The other is where the receive electrode is moving sideways relative to the line between the ground electrode and the drive electrode. These situations cause the potential to drop even though the distance between the ground and the drive electrode has not changed.

It is advantageous to calculate an electrode position in three-dimensional space. Using gradient or the slope of the rate of change of the AC signal is an important approach to gaining that position data. As an example of how this approach would be undertaken in one dimension, see FIG. 3A. An electrode at location 1 is moving to location 2. As the electrode moves gradually from left to right, the slope of the AC potential as well as the value of the AC potential are recorded. As the electrode moves somewhat to the right, its distance is measured. using the slope and the amplitude. The slope is measured by having closely spaced electrodes that are diametrically opposed in two different dimensions. As the differential voltage is measured across those closest spaced electrodes, the gradient is determined. As the electrodes move from left to right, their slope and the amplitude are determined. When the electrode moves to the right, the amplitude will change. Based on the slope, the effective distance is computed as the electrode moves from location 1 to location 1a, to location 1b, and eventually the full distance to location 2. The combination of slope and value is gradually integrated to get to location 1 and location 2.

As shown in FIG. 3B, the electrode starts at location 3 and moves over to location 4. At location 3 the slope is positive. As the drive electrode is approached, the AC potential increases. As the electrode proceeds to the right, the value increases. The slope reverses, decreasing until the electrode reaches location 4. There, the slope is flat. Eventually the slope starts increasing. The distance from location 3 to location 4 is computed simply by calculating the slope and the change in potential as the electrode position moves through the curve system.

The above explanation is demonstrative only. The actual calculations in a specific application are not necessarily as simple as the demonstrative example, which shows the distance between two electrodes in two dimensions. In the body, these fields occupy three dimensions.

In order to more rigorously determine a given electrodes' location, three different orthogonal fields are created. Fields which are not completely orthogonal but have some orthogonal nature can also be appropriate for this application. Each of these fields is provided in a different frequency. Employing a combination of slope and value in each of the frequencies allows calculation of the exact location of the electrodes.

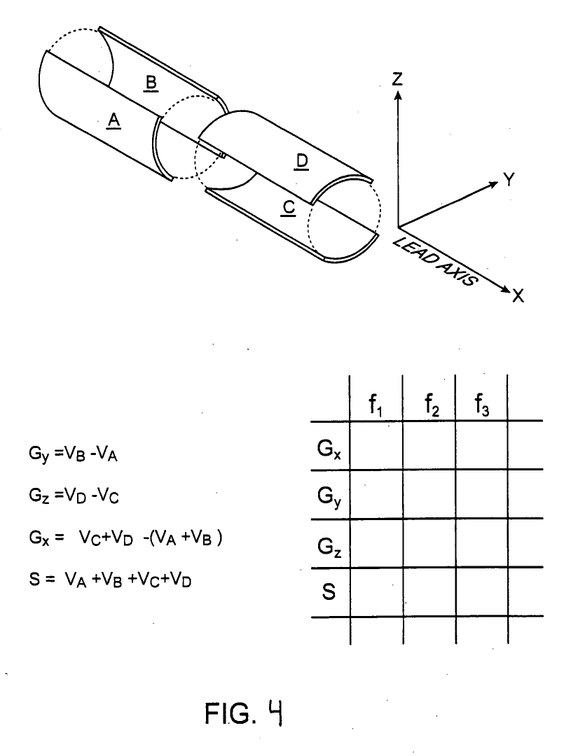

The design of one appropriate device for measuring the gradient and value of potential is shown in FIG. 4. Four electrodes are shown. Electrodes A and B are on opposite sides of the lead. Electrodes C and D are opposite from each other, but oriented 90 degrees apart from electrodes A and B. Axis X is positioned down the length of the axis of the lead body housing the four electrodes. Axis Y, perpendicular to axis X, goes through electrodes A and B. Axis Z, perpendicular to both axis X and axis Y, runs though the centers of electrodes C and D. Additional electrode configurations of interest are disclosed in PCT Patent Application Serial No. PCT/US2005/046811 titled “Implantable Addressable Segmented Electrodes,” and filed Dec. 22, 2005; the disclosure of which is herein incorporated by reference. To determine the gradient in axis Y, the AC voltage at electrode B is determined. AC voltage at electrode A is subtracted from the AC voltage at electrode B. The resulting absolute number is proportional to the gradient of the change in electrical potential and its changes over that dimension. In this case, that would be about 2 mm.

This analysis procedure is summarized as:
G_{y}=V_{B}−V_{A}

To determine the gradient in axis Z, the voltage at electrode D is determined. The voltage at electrode C is subtracted from that voltage. In both of these cases, the subtracting voltages is typically accomplished with a instrumentation amplifier. The amplifier takes the difference of the two voltages, and amplifies the difference by a factor, by example 1000. The signal is put into a lock-in amplifier. As a result, the noise from other signals is removed and only the value at the frequency of interest is recorded.

This analysis procedure is summarized as:
G_{Z}=V_{D}−V_{C}

To determine the gradient along the lead axis, voltages at electrodes C and D are added. The sum of the voltages of electrode A and B are subtracted from this number. This calculation provides the gradient in the X direction, that is the difference going along axis X of the lead.

The value of the field at that frequency is determined by the sum of these voltages, that is voltage A plus voltage B plus voltage C plus voltage D. In practice, three different pairs of drive electrodes are located along different axis. Ideally, these electrode pairs would have three different orthogonal axis. One pair of these electrodes generates a gradient for each of those frequencies. This produces a gradient in the Y direction for frequency 1, a gradient in the Y direction for frequency 2, and a gradient in the Y direction for frequency 3. These values are all calculated simultaneously because lock-in amplifiers are employed for each of those three frequencies.

This analysis procedure is summarized as:
G_{X}=V_{C}+V_{D}−(V_{A}+V_{B})

FIG. 4 provides a table of gradient and frequency to better demonstrate these concepts, and provide one structure among many appropriate structures, for assessing the sum of the values. This approach is useful where three frequencies are broadcast from pairs of electrodes that are orthogonally placed relative to each other.

From these four electrodes, four values can be computed. These values are a gradient in the X direction, a gradient in the Y direction, a gradient in the Z direction, and the sum of all of them, which would be the value of that frequency at that location. This analysis procedure is summarized as:
S=V_{A}+V_{B}+V_{C}+V_{D}

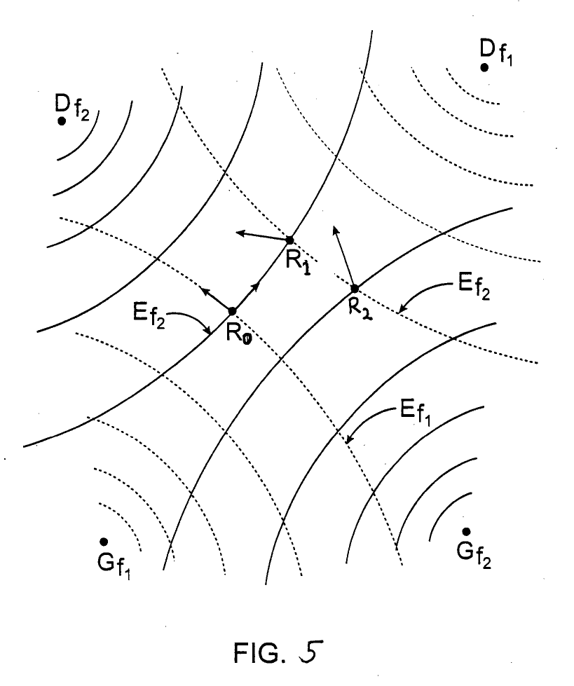

FIG. 5 shows two pairs of drive electrodes operating at two different frequencies. The ground frequency G_{f1} is shown in the lower left hand corner, and drive frequency D_{f1} is shown in the upper right hand corner. The equal potential lines are shown in dashed lines. Drive frequency D_{f2} is in the upper left hand corner. Ground frequency G_{f2} is in lower right hand corner. The equal potential lines of that frequency are shown in solid lines.

If the electrode is located conveniently at the intersection of two of these lines, the gradient at each of those frequencies can be measured. This gradient is provided as a vector of equal potential in each of these frequencies. The receive electrode at location R₀ bears an arrow that is perpendicular to the equal potential lines of frequency f₁ and a black arrow which represents the vector pointing in towards the increasing potential of frequency f₂.

From the value and the gradient, the distance is determined. By example, the electrode is located at a position along equal potential line E_{f1}. The electrode is also on the equal potential line E_{f2} which are perpendicular to the electrode. From those two numbers, the electrode's location in space is determined.

As the electrode moves in space to another position, successive measurements are taken. The electrode moves to location R₁ from original location R₀. When the electrode is at location R₁ the gradient, that is the value of drive frequency f₂, has not changed. It is still on the same potential as drive frequency f₂. The gradient has changed direction slightly, and angle has changed so that it is still pointing towards drive frequency D_{f1}. The angle is slightly different, but otherwise it has not changed much.

On the other hand, with respect to drive frequency f₁, the electrode has moved from equal potential line E_{f1}, to equal potential line E_{f2}. As that gradient is known, the distance from original location R₀ to location R₁ is calculated directly. This is accomplished by changes in slope as it goes from original location R₀ to location R₁. This is similar to the one dimensional case described in the first set of figures. If the electrode then moves to location R₂, the gradient is in frequency f₂, the angle has changed again, and the value has changed significantly.

However, since the electrode has moved along the equal potential line E_{f2}, it has not changed potential in frequency f₁. From this it is computed that the electrode is going along the gradient of the second frequency. The distances of location R₁ and location R₂ are computed in a manner similar to that demonstrated in the one dimensional drawings discussed above. From these, a matrix of the gradients and values are computed. The locations of each of the electrodes is determined by methods similar to those described herein.

The different electrical gradient tomography embodiments of the present invention have common characteristics. There are two oppositely located pairs of electrodes whose positions are at 90° from each other. From those four electrodes, the electrical gradient in three dimensions, that is X, Y and Z, are computed. The absolute value of the electrodes is also computed at multiple frequencies, shown here as frequencies F1, F2, and F3.

From those 12 values of gradients, and values at three different frequencies, a signal change is developed that produces the location of that position within the body. As these values change, the motion from one location to another location is also measured.

FIG. 5 provides a simple example of this inventive embodiment in two dimensional space, where these teachings are readily adapted by those of skill in the art to three dimensional space.

Tetrahedral Electrode Tomography

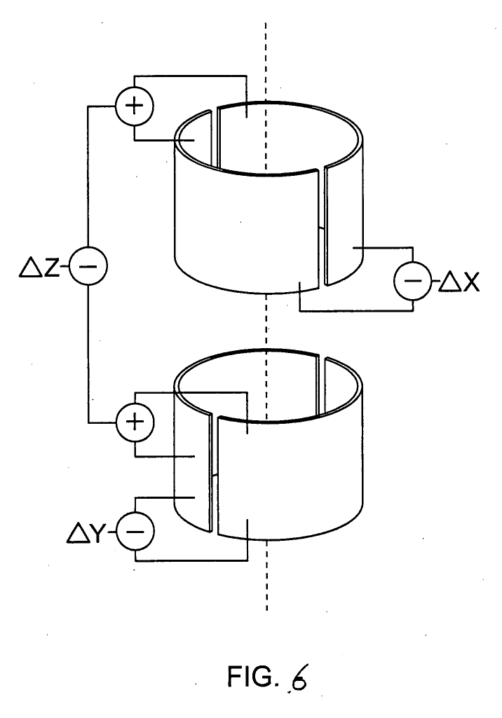

In one group of electrical tomography embodiments of the present invention, four electrodes are arranged in a tetrahedral configuration. These arrangements, such as those shown in FIGS. 6 to 8, allow an advantageous technique for dynamic, instantaneous calibration of the sensitivity of an electrical tomography device. In using a tetrahedral electrode tomography approach, a conversion factor between volts and millimeters or other units of distance is employed.

The tetrahedral electrode embodiment of the present invention allows calculation of absolute distance measurements using electrical tomography. Particular lead configurations such as those shown in FIGS. 6 to 8 provide sense gradients in the electric field at known separations of distance. Using these gradients, the signal in volts is converted by the system to provide meaningful physical numbers, such as millimeters.

The tetrahedral electrode tomography embodiment of the present invention is in some ways analogous to the system described for electrical gradient tomography. In the case of electrical gradient tomography, the gradient of an electric field is sensed in three directions. This data allows localization of the source. In tetrahedral electrode tomography, the gradient of the electric field is also sensed in three directions. This feature of the tetrahedral electrode tomography method allows proper scaling of the electric field.

As shown in FIG. 6, four electrodes situated on two electrode rings are provided. The two electrode rings are typically set sequentially within a cardiac lead. The two electrode rings are situated generally on a common axis. In this case, the electrode sets on the two rings are off-set around the common axis. This specific electrode configuration, as well as many others appropriate to this application, allows that all three gradients of the electric field are measured by taking suitable sums and differences.

The two upper electrodes are utilized to determine the X gradient. By measuring the difference between the voltages of the two upper electrodes, the X gradient in volts is calculated. Because the two upper electrodes are separated by a known distance due to this construction, the gradient in volts per millimeter is determined. The X gradient number can be applied to any electric field measured along that direction to determine absolute distances.

Also as shown in FIG. 6, the Y gradient can be determined by measuring the distance between the two lower electrodes. The Z gradient can be determined by measuring the distance between the top bi-electrode ring and the bottom ring. That is, the two semicircular electrodes summed together in each respective bi-electrode ring.

The data so obtained can usefully be expressed in a matrix, providing a distance metric. In this case, the metric tensor is provided in the rigorous sense as defined in tensor calculus. This metric tensor can then be integrated along a path as the lead moves. The metric tensor may vary as the lead moves. By integrating this data, the absolute displacements of the lead is measured.

One approach to providing the desired data is provided as follows:
S²∫g_{μv}dx^{μ}dx^{v}

Where S is the distance traveled by the lead, g_{μv} is the metric tensor, and the x^{μ} and x^{v} are the coordinate directions.

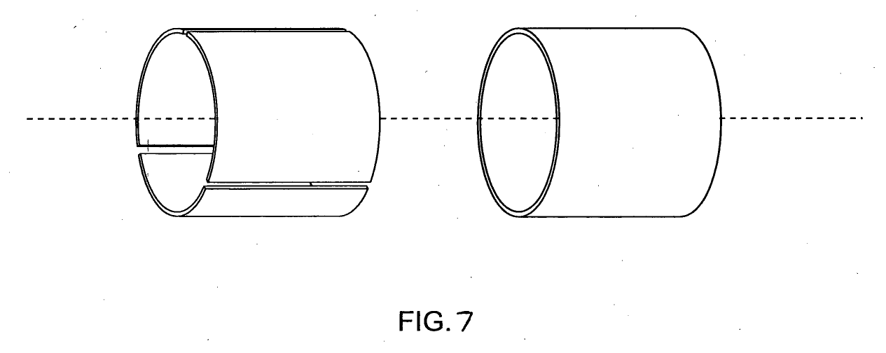

Two additional embodiments are provided in FIGS. 7 and 8. These electrode configurations provide alternate methods of constructing the distance metric. FIG. 7 consists of a triplet of electrodes around the circumference, and a fourth electrode displaced distally along the lead. By forming appropriate algebraic combinations of the measurements between the electrodes, the three components of the distance metric, the X, Y and Z gradients, are determined.

FIG. 8 shows an additional configuration which is based on an approach similar to that shown in FIG. 7. In the case of the configuration shown in FIG. 8, the X, Y, and Z gradients are determined directly by taking appropriate differences of pairs of electrodes.

Processing of Data

The ET data obtained using the present methods may be employed as raw data or processed in various ways, as desired. For example, using either internal or external orthogonally applied electrical fields, a value for voltage at a tissue location (e.g. an electrode on a cardiac lead, or an epicardial lead) can be obtained to determine a change of voltage. From the voltage data a position signal can be calculated for a location (e.g. an electrode, or a tissue location), and by evaluating the rate of change of the position signal, the position as a function of time can be determined (e.g. the duration of the cardiac cycle). In certain embodiments, at least one of the position signals calculated can be a baseline position signal. In certain embodiments, the position signal can be calculated after an intervention (e.g. a paced position signal, as when employing CRT). In certain embodiments, two or more position signals can be calculated under different conditions (e.g. at baseline, and after pacing with CRT). The position signal(s) can be calculated from a single cardiac cycle, or can be calculated from data averaged over several cardiac cycles, e.g. one cardiac cycle, two cardiac cycles, or three or more cardiac cycles.

The position of a second tissue location (e.g. a second electrode on the same cardiac lead, or an electrode on a separate lead) as a function of time can also be determined by measuring the voltage at that electrode, and the motion at a second tissue location can be compared to motion at a first tissue location. The position of a third, a fourth, a fifth, or more tissue locations (e.g. additional electrodes on the same cardiac lead, or electrodes on a separate lead) as a function of time can also be determined by measuring the voltage at each electrode, and the motion at each tissue location can be compared to motion at other tissue locations.

The position signal can be calculated by separating the monitored voltage data into a cardiac component, an interference component and a noise component. At least one contributor to the interference component is interference from respiration. In some embodiments, calculating the position signal comprises removing the respiration interference component of the measured voltage in order to obtain a position signal. The respiration interference component can be identified and removed in post-processing in order to remove its effect on the position signal generated by cardiac motion. In other embodiments, the respiratory signal can be identified and isolated, and used to compare data sets obtained at the same point in the respiration cycle, usually at end-expiration.

Where desired, the cardiac component data can be normalized, e.g., to increase the accuracy of the position data calculated from the voltage data. Techniques for normalizing the data may include assigning scale factors to signals obtained from a sense electrode to correct for distortions in the electric field. In one embodiment, predetermined scale factors, e.g., based on physiologic characteristics, e.g., the height and weight of the subject, may be employed. In another embodiment, the scale factors can be dynamic, meaning that the scale factors can change over time (e.g. at different points in the cardiac cycle, or from one cardiac cycle to the next) based on changes in the ambient electric fields (e.g. changes in strength, gradient, or direction of the electric field(s) surrounding the sense electrode). In one embodiment, scale factors can be based on a known inter-electrode distance for two or more electrodes that are located in the field, e.g. a one centimeter known separation between two electrodes on a lead, may be employed, where these dimension-based scale factors may be used to correct measurements for the remaining electrodes. In this embodiment, electrodes in close proximity (e.g. 1 cm apart) are electrically coupled. When the lead is bent, the distance between the electrodes decreases thereby changing the electrical coupling. The measured electrical coupling signal provides data related to bending of the lead in the region around the electrodes. This data can be used to normalize signals from the remaining electrodes. A third method involves directly measuring distortion in the electric field to obtain a scale factor, e.g., by using a segmented tetraelectrode as described in United States Provisional Application Ser. No. 60/790,507 titled “Tetrahedral Electrode Tomography,” and filed Apr. 7, 2006; the dislcosure of which is herein incorporated by reference.

Devices and Systems

In certain embodiments, devices and systems are employed for practicing the ET methods. The system of certain embodiments is made up of the following main components or devices: 1) one or more electrodes with at least one electrode (e.g., the sensing electrode) being stably associated, at least temporarily, with a heart wall, where the heart wall location may be an intracardial or epicardial location, as desired and depending on the particular application; 2) a signal generator; 3) a signal receiver (where the signal generator and receiver work together to produce the applied electric field; 4) a signal processor; and 5) a signal display. For CRT applications, in order to optimize CRT in real-time, the electrodes can alternate back and forth between pacing and motion sensing functions.

In certain embodiments, the sense electrode(s) is present on a medical carrier, e.g., lead. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 or DF-1 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, a particular configuration, e.g., S-bend, etc. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by one or more of the connector arrangements described above.

This approach can be extended to pacing leads with a plurality of sensing electrodes placed around the heart, which provides a more comprehensive picture of the global and regional mechanical motion of the heart. With multiple electrodes, artifacts such as breathing can be filtered out. Furthermore, multiple electrodes can provide three-dimensional relative or absolute motion information by having electrodes switching between the roles of reference, driver, or sense electrode. A multi-electrode lead, such as a multiplex lead can be used, or multiple electrodes can be present on a guidewire, for example. Indeed any of the electrodes (including a pacemaker can) in this system can be used as a reference, driver, or sense electrode.

This approach can be further extended to employ a variety of electrical field generating elements, creating distinct electrical fields in each of multiple planes, or axes. Sensing electrodes can simultaneously report amplitude from each of the multiplanar electrical fields, thereby improving resolution in characterizing intracardiac wall motion. In one embodiment, three essentially orthogonal fields can be created using internal and/or external field generating elements. For example, the fields can be created with X, Y, and Z axes such that the “X” electric field is oriented in a right/left direction with respect to a patient; the “Y” electric field is oriented in a superior/inferior direction with respect to a patient; and the “Z” electric field is oriented in an anterior/posterior direction with respect to a patient. The three essentially orthogonal fields can also be oriented such that they are aligned with principle axes of the heart, such that a first plane or axis is parallel to the long axis of the left ventricle (“long-axis plane”), a second plane is oriented perpendicular to the first (“short-axis plane”), and a third plane is perpendicular to both the long- and short-axis planes (“four-chamber plane”). Using such resolution-enhancing embodiments can, with proper calibration, yield parameters, including stroke volume and ejection fraction, which are important in CHF management, e.g., as further developed below.

In practicing the subject methods, an electric field can also be generated such that the voltages applied to two or more electrodes can be adjusted to synthesize a “virtual electrode,” such that the effective position the electric fields return to is not coincident with either electrode. For example, if three electrodes are positioned at the vertices of an equilateral triangle, and one of the electrodes is selected as ground, while the other two electrodes are energized at the same voltage, the effective direction of the field will be from the ground electrode to a point halfway between the two positive electrodes. By varying the relative voltages on the positive electrodes, the direction of the field can be “steered” or oriented to a direction that falls between the two electrodes. By moving the ground electrode, or by varying the voltage on one, two, or all three electrodes, for example, the direction of an electric field can be oriented in a direction of motion of interest. In certain embodiments, the electric field(s) can be reoriented at least once over a given period of time.

The precision of the “steering”, or the ability to select the direction of the electric field, can be increased by adding more electrodes (e.g. around a ring external to the body, or on a lead). In one embodiment, a belt with many segmented electrodes can be placed around the chest of a subject. By choosing the appropriate linear combination of voltages on the segments, a relatively flat electric field can be generated in an arbitrary orientation. In certain embodiments, a single electric field is generated, and in some embodiments, two fields that are substantially orthogonal over a large area can be generated. In certain embodiments a plurality of different electric fields can be generated, e.g., two or more, such as three or more, e.g., four or more, six or more, etc., where in certain of these embodiments, the generated electric fields may be substantially orthogonal to one another.

Another extension of this approach is to generate more than one electrical field in each plane through the use of the several driving electrodes. Several fields of different frequency can be superimposed in the same configuration. In this application, each co-planar electrical field would be tailored to exploit different propagation characteristics within the human body. In this way, in addition to wall motion, valuable information can be obtained about the composition of the local fluids and tissues. Such data will prove clinically important in determining, without limitation, a variety of different physiological parameters, such as but not limited to: pulmonary congestion, myocardial thickness and hemodynamic parameters such as ejection fraction, as further developed below.

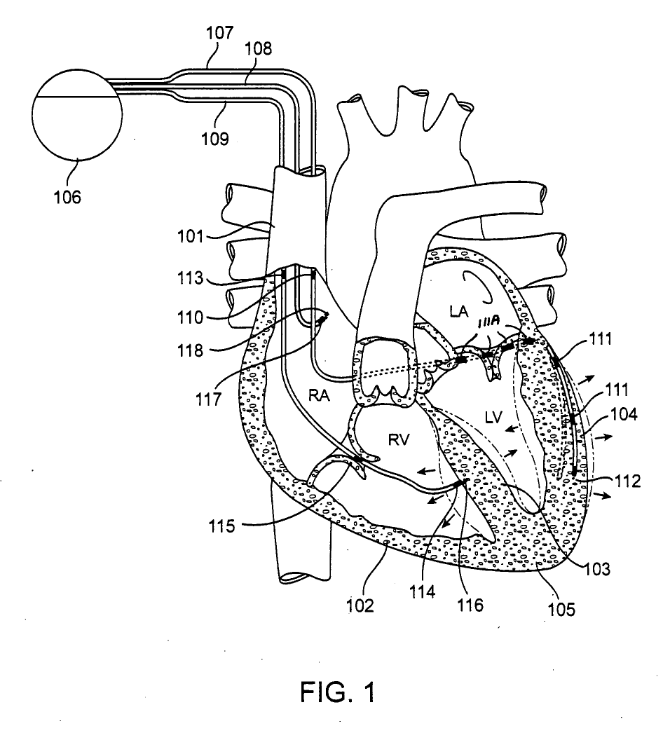

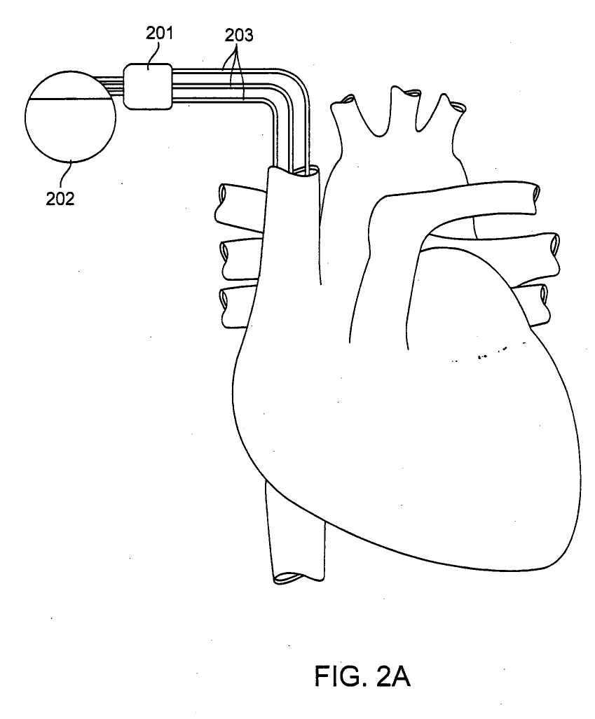

FIG. 1 provides a cross-sectional view of the heart with of an embodiment of the inventive electrical tomographic device, e.g., as embodied in a cardiac timing device, which includes a pacemaker 106, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more electrodes 110, 111, 112, and 111A. The distal electrodes 111 and 112 are located in a left ventricular cardiac vein and provide regional contractile information about this region of the heart. Also shown are four electrodes 111A in the coronary sinus, in the region of the mitral annulus. The most proximal electrode 110 is located in the superior vena cava in the base of the heart. This basal heart location is essentially unmoving and therefore can be used as one of the fixed reference points for the cardiac wall motion sensing system.

Once the electrode lead 109 is fixed on the septum, electrode lead 109 provides timing data for the regional motion and/or deformation of the septum. The electrode 115 which is located more proximally along electrode lead 109 provides timing data on the regional motions in those areas of the heart. By example, an electrode 115 situated near the AV valve, which spans the right atrium in the right ventricle, provides timing data regarding the closing and opening of the valve. The proximal electrode 113 is located in the superior vena cava in the base of the heart. This basal heart location is essentially unmoving and therefore can be used as one of the fixed reference points for the cardiac wall motion sensing system.

The electrode lead 108 is placed in the right atrium using an active fixation helix 118. The distal tip electrode 118 is used to both provide pacing and motion sensing of the right atrium.

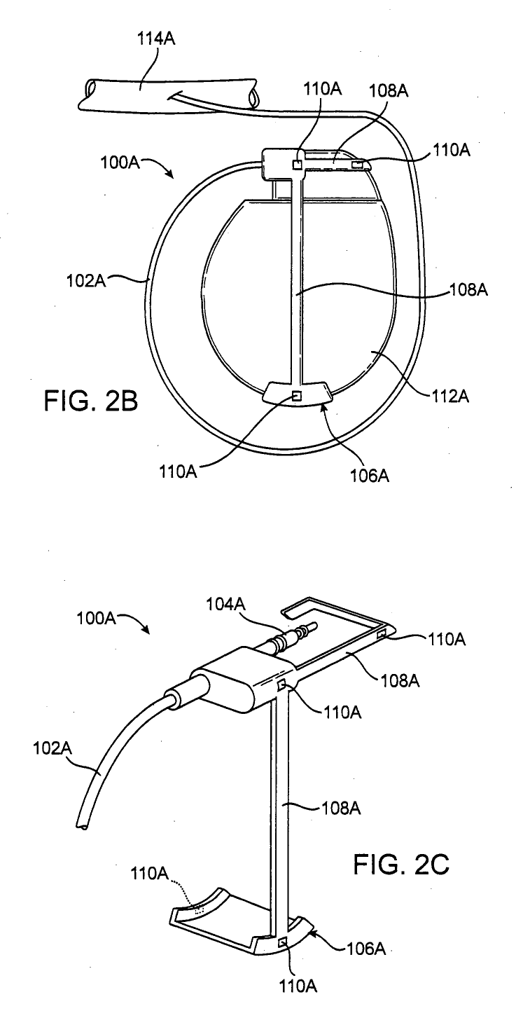

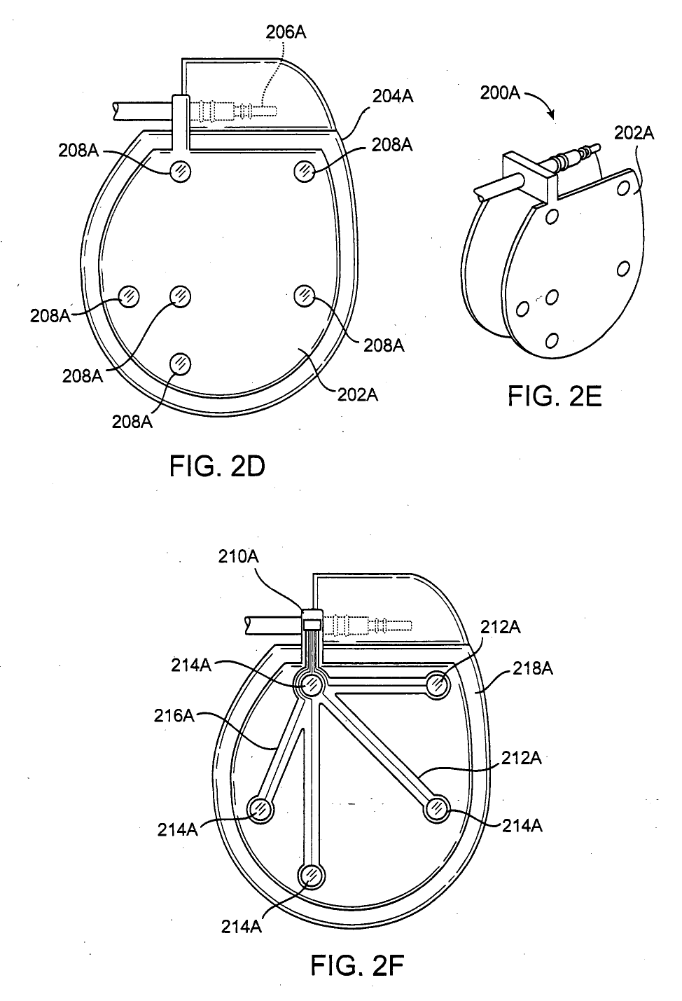

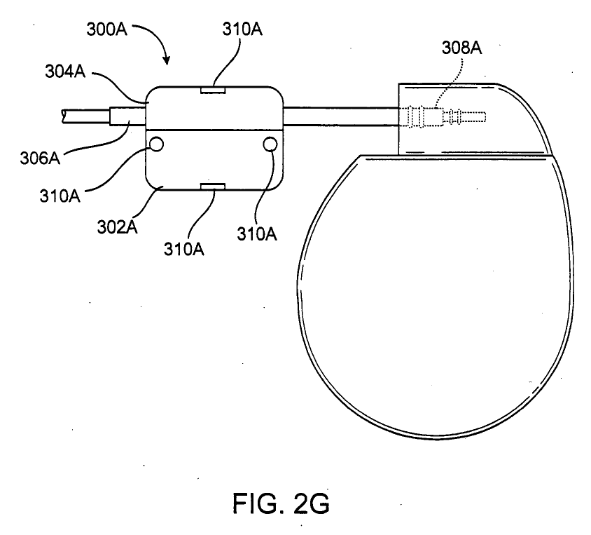

FIG. 2A provides a view of an additional of the embodiment described in FIG. 1 with an add-on module 201 which is connected in series in between pacemaker 202 and the electrode leads 203. The add-on module (i.e., adaptor) is comprised of a hermetically sealed housing which contains all the software, hardware, memory, wireless communication means, and battery necessary to run the cardiac wall motion sensing system. The housing is made of titanium and can be used as the reference electrode. On the proximal end, the add-on module 201 has lead type proximal connectors which can plug into the pacemaker header. On the distal, the add-on module 201 provides connectors for electrode leads 203. One of the main advantages of this embodiment is that it can be used with any commercial pacemaker. Even patients who already have a pacemaker and lead system implanted can benefit from this add-on module 201. In an outpatient setting and using a local anesthetic a small incision is made expose the subcutaneously implanted pacemaker. The leads 203 are then disconnected from the pacemaker and connected to the add-on module 201 which in turn is plugged into the pacemaker header. The incision is then sutured close and the patient can now immediately benefit from the cardiac motion sensing system.

Another embodiment of an add-on module is depicted in FIGS. 2B to 2G, which module provides for one or more additional electrode sites, where the add-on module can be configured, as desired, to be employed with other implantable devices, such as pacemakers, to provide for the electrode field(s) desired for a given application. The electrode add-on module can include one or more electrodes, e.g., 2 or more, 3 or more, 4 or more, 5 or more, etc., as well as electrode pairs, e.g., 2 or more pairs, 3 or more pairs, 4 or more pairs, 5 or more pairs, as desired. Typically, the add-on module is configured or designed to be implantable, e.g., in a convenient subcutaneous location, and in certain embodiments may be configured to associate with, e.g., attached to, snap on to, etc., another implantable device, such as a pacemaker. As such, embodiments of the add-on modules provide additional electrode sites within the subcutaneous area near the pacemaker, and can be very easily and quickly placed during the implantation procedure.

In one representative as shown in FIGS. 2B and 2C, the device 100A is comprised of an electrode lead 102A inserted into a subclavian vein 114A with on the proximal end an IS-1, IS4 or other connector 104A and a multielectrode clip-type device 106A with flexible struts 108A. The electrodes 110A can be positioned on all sides of the pacemaker 112A can to generate electrical fields in any direction for the ET method described previously. One advantage is that the position of all the electrodes 110A relative to each is fixed and known. Furthermore, the anatomical location of the device 100A is quite repeatable from one patient to the next which will mitigate variability of the ET system between patients. In addition, the electrodes 110A, being located in a subcutaneous pocket, are removed from the problematic flow velocity induced changes in blood conductivity that affect electrical fields generated by the intravenous, atrium and ventricle electrodes. Also, the device 100A can be easily and quickly clipped directly onto the pacemaker to stabilize it.

In another representative embodiment shown in FIGS. 2D and 2E, the device 200A is comprised of a low profile device 202A which slides into place around the front and/or back of the pacemaker 204A with minimal addition to the pacemaker volume. The IS-1, IS4 or other connector 206A provides stability. The front and back portions include one or more electrodes 208A are used to generate electrical fields.

In another representative embodiment shown in FIG. 2F, the device 210A is also comprised of a very low profile “flex circuit” type device 212A with multiple electrodes 214A and conductors 216A , where the device is placed on and is connected to the pacemaker can 218A.

In another representative embodiment shown in FIG. 2G, the device 300A is comprised of a housing 302A containing electronics, RF telemetry, and battery, and a header 304A for the connectors of the electrode lead 306A and pacemaker can 308A. On the outside of the housing are located multiple electrodes 310A to generate multiple electrical fields. In certain embodiments, this device could be used with standard leads, Protoplex™ leads, standard pacemakers, and/or ET enabled pacemakers.

The add-on modules of these embodiments can, in addition to providing one or more additional electrodes, be a platform device for various sensors such as temperature sensors, pressure sensors, and biosensors, as desired.

Additional device configurations may be found in PCT application serial no. PCT/US2005/036035 titled “Continuous Field Tomography,” and filed on Oct. 6, 2005, the disclosure of which is herein incorporated by reference.

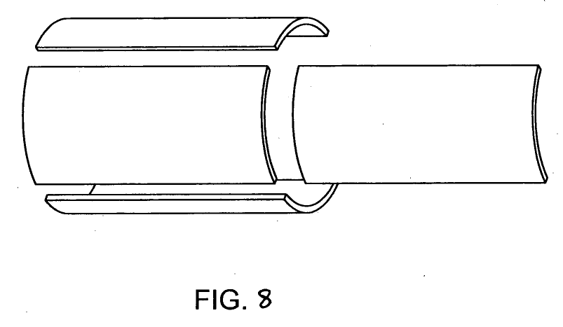

An example of an electrical tomography system according to an embodiment of the present invention is shown in FIG. 9. The embodiment depicted in FIG. 9 is configured to use the electrical tomography technique to measure dysynchronous cardiac motion and assist in optimizing cardiac resynchronization therapy (CRT) for congestive heart failure (CHF) patients as described in this patent application. In FIG. 9, the device is comprised of an electrical tomography system 9000 includes hardware and software for generation of electrical fields, cardiac pacing, data acquisition, data processing, and data display; a skin electrode cable 9002 which is connected to three pairs skin electrodes (right/left torso, chest/back, and neck/leg) which are used to generate three orthogonal electrical fields across the heart; a cardiac electrode cable. 9004 which is connected to the internal electrodes within the heart; a guide catheter 9014 which is inserted into the subclavian vein and used to access the coronary sinus; one or more multielectrode guidewires/minicatheters 9018, 9022, and 9024 which have multiple electrodes at the distal end and are inserted via the guidecatheter 9014 into the main cardiac vein and its sidebranches such as the lateral and postero-lateral cardiac veins; and a standard RV lead 9024 with an active fixation helical electrode 9024 attached to the septal wall.

One embodiment of procedural steps would be as follows. The three pairs of skin electrodes are placed on the patient to create the three orthogonal electrical fields spanning the heart. See FIG. 11. The skin electrode cable 9002 is used to connect the skin electrodes to the electrical tomography system 9000. Under sterile field the physician inserts via the subclavian vein an RV lead into the right ventricle and screws the active fixation helical electrode into the septal wall. The physician then uses the guide catheter 9014 to cannulate the coronary sinus. A venogram using a balloon catheter inserted through the guidecatheter 9014 is performed to map the cardiac vein anatomy. The multielectrode guidewires 9018, 9020, 9022 are inserted into the guide catheter 9016. The first multielectrode guidewire 9022 is advanced into the great cardiac vein along the septum until it reaches the apex of the heart. This multielectrode can in addition to the RV electrode lead be used to track the motion of the septal wall. The second multielectrode guidewire 9020 is steered into one of the lateral cardiac veins of the left ventricle. And the third multielectrode guidewire 9018 is steered into one of the postero-lateral cardiac veins of the left ventricle. The cardiac cable 9004 is plugged into the electrical tomography system 9000 and connected to the proximal connectors 9008, 9010, 9012 of the multielectrode guidewires 9018, 9020, 9022, and the proximal IS-1 connector 9006 of the RV electrode lead 9016.

Once all the devices are in place and connected, the three orthogonal electrical fields are turned on and a baseline measurement of the measured motion of all the electrodes is recorded. The amount of baseline intraventricular dyssynchrony is calculated by comparing the motion of the electrodes in the lateral and postero-lateral cardiac veins (multielectrode guidewire 9018, 9020) and the electrodes along the septum (RV lead distal electrode 9024 and/or multielectrode guidewire 9022). Next, CRT test is initiated by performing biventricular pacing with the RV lead distal electrode 9024 and one of the LV electrodes in the lateral or postero-lateral cardiac veins (multielectrode guidewire 9018, 9020). Biventricular pacing is repeated with each of the LV electrodes one by one (multielectrode guidewire 9018, 9020) while recording the corresponding intraventricular dyssynchrony indices. It is important to note that while the LV pacing location is being changed with each test, the motion sensing electrodes used to measure the intraventricular dyssynchrony are not changing position relative to the heart. This allows direct comparison of intraventricular dyssynchrony measurements between all the tests. The data from all the tests is used to generate a map of the optimal LV pacing sites for CRT, thereby identifying the best cardiac vein for placement of the LV electrode lead.

At this point the multielectrode guidewire which is located in the selected cardiac vein is left in place while all the other ones are pulled out. The proximal connector 9008, 9010, or 9012 of the multielectrode lead left in place, is removed and the implantable LV electrode is inserted over-the-wire into the selected cardiac vein and positioned under fluoroscopy to match the position of the determined ideal LV pacing site. In the case of implantation of the multielectrode lead, position within the selected cardiac vein is not critical because of the flexibility provided by the multiple electrodes along the lead.

In another embodiment, at this point all of the multielectrode guidewires are removed and under fluoroscopy the LV electrode lead is positioned using standard lead delivery tools to match the position of the most ideal accessible LV pacing site. Finally, the standard CRT implantation procedure is resumed.

In certain embodiments, a plurality of drive electrode pairs are present, each generating a distinct electric field, where the fields are generally oriented along different endocardial planes, e.g., as may be generated by the different driving electrode pairs shown in FIG. 11. Representative planes generated in certain embodiments are between relatively immobile electrodes located in the superior vena cava, the coronary sinus and an implantable pulse generator in the left or right subclavicular region. Additional electrode locations include the pulmonary artery, and subcutaneous locations throughout the thorax, neck and abdomen, as well as external locations.

In certain embodiments, additional planes are generated from electrodes experiencing relatively greater motion than those already described (e.g., right ventricular apex, cardiac vein overlying left ventricle, etc.). In certain embodiments, to obtain absolute position, computational techniques are employed with reference to other available planes in order to eliminate the motion component of the drive electrodes with respect to the sense electrodes. In certain applications of the system, relative timing and motion information is of greater importance than absolute position. In these applications, at least, significant movement of one or more electrical field planes may be tolerated with minimal or even no real-time computation intended to compensate for this motion.

Another embodiment of the present invention provides a system configured for use in analyzing cardiac motion. During operation, the system places “n” cardiac electrodes and applies an AC voltage to a tissue region where the cardiac electrodes reside. The system then detects an induced voltage on each electrode and constructs an n×n correlation matrix based on the induced voltage on each cardiac electrode. The system subsequently diagonalizes the correlation matrix, thereby solving for eigenvalues and eigenvectors of the correlation matrix.

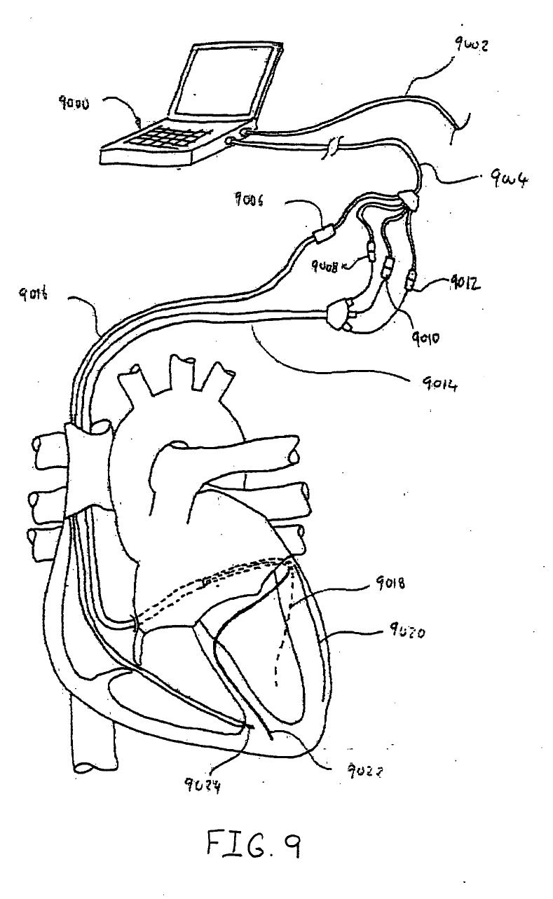

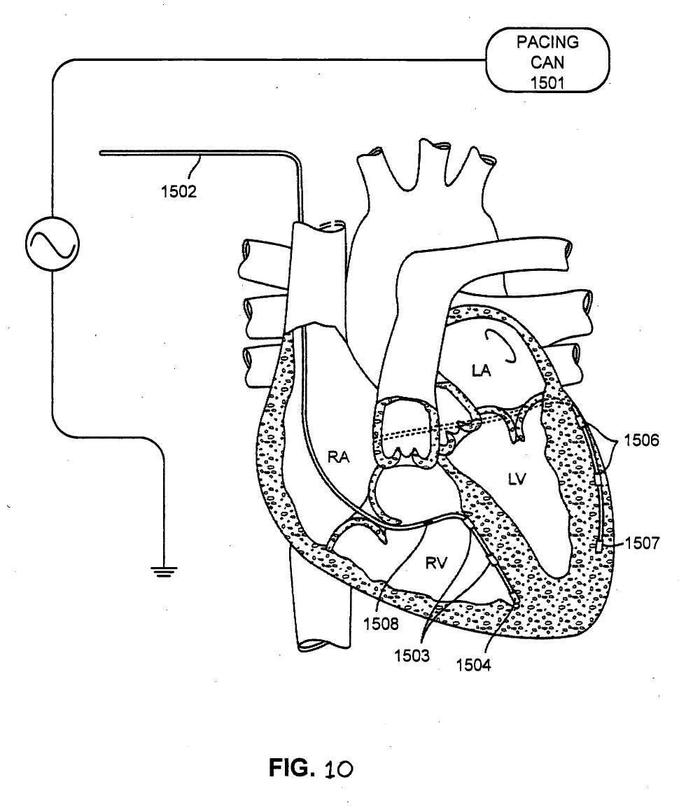

FIG. 10 illustrates an exemplary configuration for electrical tomography of cardiac electrodes, in accordance with an embodiment of the present invention. FIG. 10 shows the locations 1503, 1504, 1506 and 1507 of a number of pacing electrodes. A pacing can 1501 resides in an external or extra-corporeal location. Pacing can 1501 may transmit pacing pulses to the electrodes through a pacing lead 1502.

Electrodes at locations 1503 and 1504 are coupled to right ventricular lead 1502, which travels from a subcutaneous location for. a pacing system (such as pacing can 1501) into the patient's body (e.g., preferably, a subclavian venous access), and through the superior vena cava into the right atrium. From the right atrium, right ventricular lead 1502 is threaded through the tricuspid valve to a location along the walls of the right ventricle. The distal portion of right ventricular lead 1502 is preferably located along the intra-ventricular septum, terminating with fixation in the right ventricular apex. As shown in FIG. 10, right ventricular lead 1502 includes electrodes positioned at locations 1503 and 1504. The number of electrodes in ventricular lead 1502 is not limited, and may be more or less than the number of electrodes shown in FIG. 10.

Similarly, a left ventricular lead follows substantially the same route as right ventricular lead 1502 (e.g., through the subclavian venous access and the superior vena cava into the right atrium). In the right atrium, the left ventricular lead is threaded through the coronary sinus around the posterior wall of the heart in a cardiac vein draining into the coronary sinus. The left ventricular lead is provided laterally along the walls of the left ventricle, which is a likely position to be advantageous for bi-ventricular pacing. FIG. 10 shows electrodes positioned at locations 1506 and 1507 of the left ventricular lead.

Right ventricular lead 1502 may optionally be provided with a pressure sensor 1508 in the right ventricle. A signal multiplexing arrangement facilitates including such active devices (e.g., pressure sensor 1508) to a lead for pacing and signal collection purpose (e.g., right ventricular lead 1502). During operation, pacing can 1501 communicates with each of the satellites at locations 1503, 1504, 1506 and 1507.

According to one embodiment, pacing can 1501 is used as an electrode to apply an AC voltage to the heart tissue. The ground of the AC voltage source may be at another location on the patient's body, for example a patch attached to the patient's skin. Accordingly, there is an AC voltage drop across the heart tissue from pacing can 1501 toward the ground location. An electrode implanted in the heart has an induced electrical potential somewhere between the driving voltage and the ground. By detecting the induced voltage on the electrode, and by comparing the induced voltage with the driving voltage, one can monitor the electrode's location or, if the electrode is moving within the heart, the instant velocity of the electrode. For example, a first signal can be detected at a first time (e.g. the position of an electrode at the beginning of systole), and then at a second time (e.g. the position of the electrode at the end of systole). The velocity can then be computed by differentiating, or taking the derivative of, the position signal of the object (e.g. an electrode). The velocity of an object (e.g. an electrode, or a tissue location) is its speed in a particular direction, or the rate of displacement, and indicates both the speed and direction of an object.

The system may also apply a direct-current (DC) voltage to the tissue. However, an AC driving voltage is preferable to a DC voltage in representative embodiments, because AC signals are more resistant to noise. Because the induced voltage signal on an electrode has substantially the same frequency as the driving AC voltage does, one can use a lock-in amplifier operating at the same frequency to reduce interferences from noise.

The system may apply the electrical field in various ways. In one embodiment, the system may use a pacing can and an existing implanted electrode, or two existing implanted electrodes to apply the driving voltage. In a further embodiment, the system may apply the driving voltage through two electrical-contact patches attached to the patient's skin.

Based on the same principle, one can apply three AC voltages in three directions (x, y, and z), which are substantially orthogonal to each other, to measure the location of an electrode in a 3-dimensional (3-D) space. FIG. 11 illustrates an exemplary configuration for 3-D electrical tomography of cardiac electrodes, in accordance with an embodiment of the present invention. The system applies an AC voltage vₓ through a pair of electrodes 1604 in the x direction. Similarly, the system applies v_{y} and v_{z} in the y direction and z direction, respectively. vₓ, v_{y}, and v_{z} each operates at a different frequency. As a result, three induced voltages are present on an implanted electrode 1602. Each induced voltage also has a different frequency corresponding to the frequency of the driving voltage in each direction. Therefore, by detecting the three induced voltages using three separate lock-in amplification modules, each of which operating at a different frequency, one can determine the electrode's location in a 3-dimensional space.

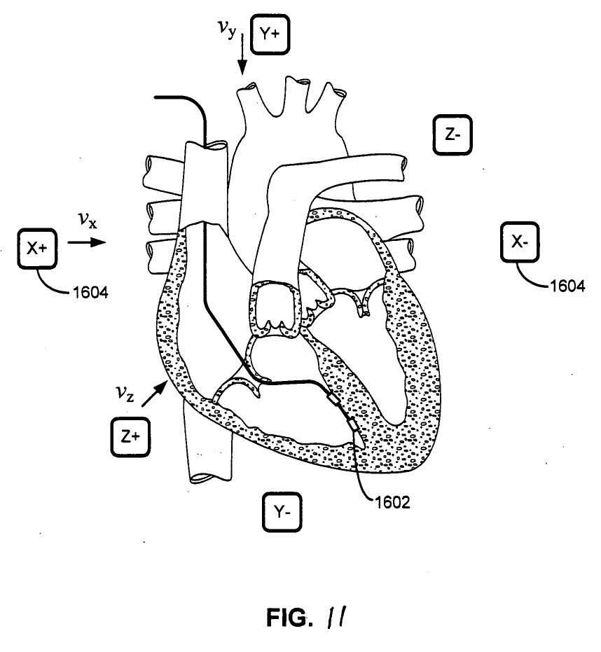

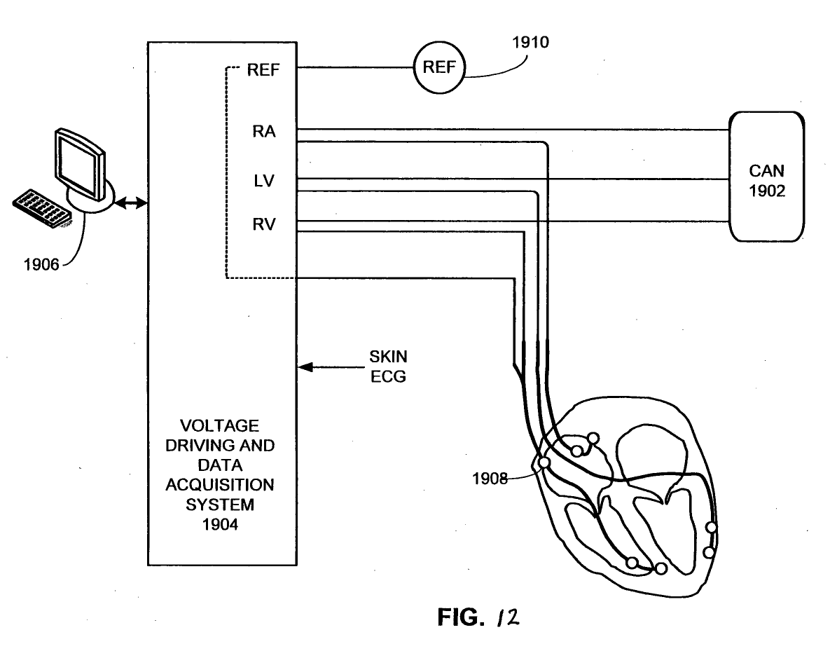

One advantage of an electrode tomography system applying an electrical field is that the system can operate on existing cardiac pacing system and, therefore, incurs minimum risk to a patient. FIG. 12 illustrates an electrical tomography system based on an existing pacing system, in accordance with an embodiment of the present invention. In this example, there are a number of pacing electrodes implanted in a patient's heart. These electrodes may be off-the-shelf electrodes for regular cardiac pacing purposes.

A voltage-driving and data-acquisition system 1904 couples to a pacing can 1902. System 1904 also couples to the electrodes which reside in the right atrium (RA), left ventricle (LV), and right ventricle (RV). Leads from pacing can 1902 are first routed to system 1904 and then routed to the electrodes. System 1904 can use the leads to drive any electrode, including pacing can 1902, and can detect induced signals on non-driving electrodes through the leads. System 1904 also has a reference port which may couple to an external voltage reference point, such as the ground. In the example in FIG. 12, electrode 1908 is coupled through the lead to the reference port, which is coupled to a ground reference voltage 1910.

The arrangement described above allows pacing can 1902 to send regular pacing signals to the electrode while performing electrical tomography. Such simultaneous operation is possible because pacing signals are typically short pulses, whereas the driving voltage is a constant sinusoidal signal with a well defined frequency. Furthermore, system 1904 may receive skin electrocardiogram (ECG) data to assist the analysis of the electrical tomography signals. System 1904 also interfaces with a computer 1906, which performs analysis based on the collected data.

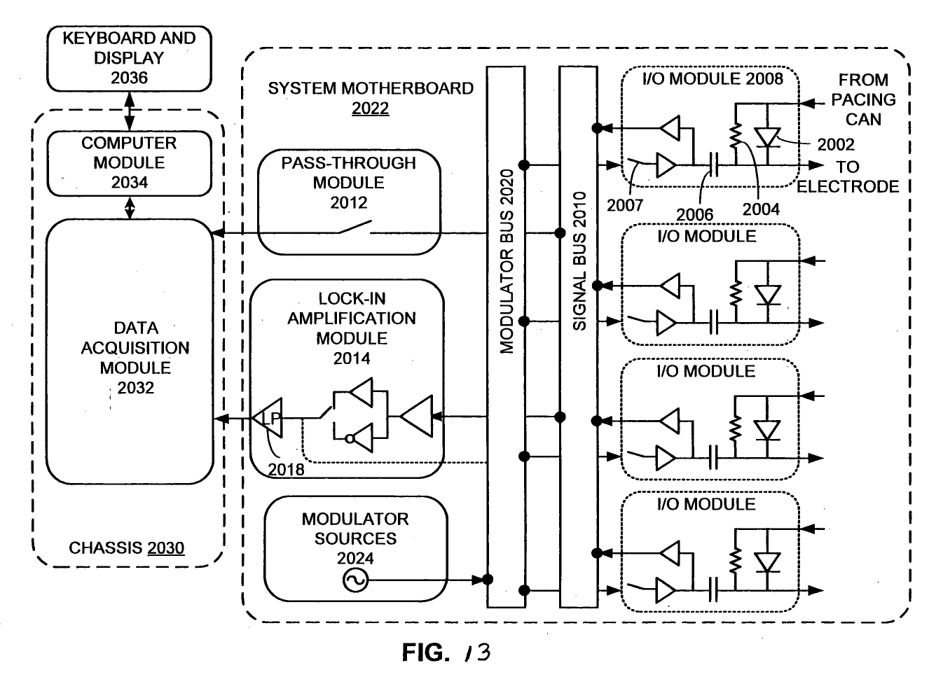

FIG. 13 illustrates a schematic circuit diagram for the voltage-driving and data-acquisition system 1904 in FIG. 12, in accordance with an embodiment of the present invention. The system includes a system motherboard 2022 and a chassis 2030. System motherboard 2022 accommodates a number of input/output (I/O) modules, such as I/O module 2008. Also included on system motherboard 2022 are a signal bus 2010, a modulator bus 2020, a pass-through module 2012, a lock-in amplification module 2014, and a set of modulator sources 2024.

An I/O module may contain a number of I/O circuits, each serving one data channel. The I/O circuit in I/O module 2008 has a loop-back stage which includes a diode 2002 and a resistor 2004. Resistor 2004 and diode 2002 allow a pacing signal from the pacing can to pass through and reach the electrode. In addition, resistor 2005 and diode 2002 serves to isolate the AC driving voltage used by the tomography system from the pacing can.

A coupling capacitor 2006 allows receipt of induced AC signals from an electrode. Capacitor 2006 also couples a driving AC voltage to an electrode when the electrode serves as a driving electrode. Correspondingly, switch 2007 is engaged when the coupled electrode is a driving electrode, and is disengaged when the coupled electrode is a sensing electrode.

When receiving signals, I/O module 2008 transmits the received AC signals to the signal bus 2010, which subsequently transmits the received signals to lock-in amplification module 2014. When used for driving an AC voltage, I/O module 2008 receives an AC voltage from the modulator bus 2020. Note that modulator sources 2024 include a number AC voltage sources and can drive multiple electrodes simultaneously. Accordingly, modulator bus 2020 is responsible for routing the AC driving voltages to proper I/O modules.

Lock-in amplification module 2014 includes multiple lock-in amplifier circuits. In a lock-in amplifier circuit, an input signal is first amplified, and then multiplied by a signal with a reference frequency to produce a product signal. When the input signal is a detected AC signal induced on an electrode, the corresponding AC driving voltage is used as the reference signal, so that the product signal has a DC component that reflects the level of the induced AC signal. The product signal is then filtered by a low-pass filter 2018 to remove any noise at other frequencies, including a pacing pulse. Furthermore, pass-through module 2012 transmits the received signals directly to data acquisition module 2032 without any lock-in amplification.

Chassis 2030 includes the data acquisition module 2032 and a computer module 2034. Data acquisition module 2032 digitizes the received signals and transfers the data to computer module 2034. Computer module 2034 may include a central processing unit (CPU), a memory, and a hard drive, and is responsible for storing and analyzing the data. A keyboard and a display 2036 interfaces with computer module 2034 to facilitate data input and output.

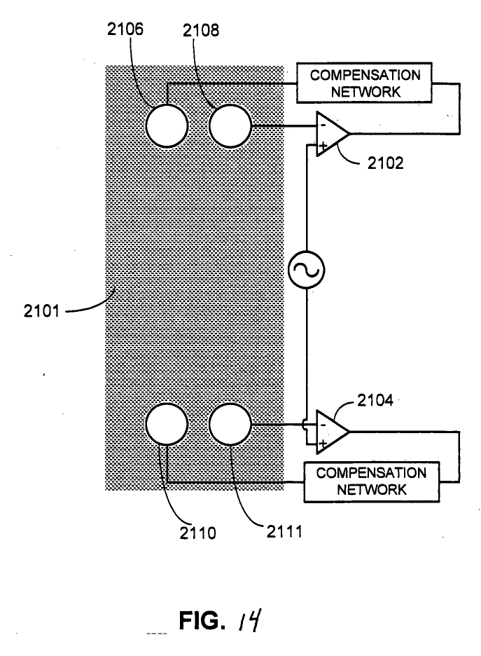

In certain applications, it may be desirable to estimate large electrode interface impedance. FIG. 14 illustrates one embodiment of the present invention that eliminates the effect of large electrode interface impedance by using four electrodes for driving an AC voltage. Two driving electrodes, 2106 and 2110, are submerged in blood (or organic tissue) 2101. Two auxiliary electrodes, 2108 and 2111, are placed in the vicinity of electrodes 2106 and 2110, respectively.

To eliminate the effect of large interface impedance of electrodes 2106 and 2110, and to obtain a stable AC voltage drop across the blood (or tissue) 2101, the system facilitates two operational amplifiers (OPAMPs) 2102 and 2104. The positive input of OPAMP 2102 is coupled to auxiliary electrode 2108, and the positive input of OPAMP 2104 is coupled to auxiliary electrode 2111. An AC voltage source is coupled between the two negative inputs of the two OPAMPs. Driving electrode 2106 is coupled to the output of OPAMP 2102. Correspondingly, driving electrode 2110 is coupled to the output of OPAMP 2104.

With this configuration, there remains a stable AC voltage drop between auxiliary electrodes 2108 and 2111, because the two inputs of an OPAMP have substantially the same electric potential. Moreover, although there is also a large interface impedance around auxiliary electrodes 2108 and 2111, there is only negligible current flowing through the two positive OPAMP inputs. Therefore, the voltage drop due to large interface impedance of auxiliary electrodes 2108 and 2111 is minimal. Consequently, the voltage drop across blood (or tissue region) 2101 remains the same as the driving AC voltage.

The voltage difference between driving electrodes 2106 and 2110, however, may not be a constant value. This is because the current flowing through the blood is kept constant (because the voltage drop between auxiliary electrodes 2108 and 2111 is constant, and because the blood impedance typically remains stable). Hence, whenever there is variation in the interface impedance of driving electrode 2106 or 2110, the voltages on these driving electrode also change correspondingly. Nevertheless, the total voltage drop across the blood region is stable, which facilitates detection of changes in an induced voltage of a target electrode whose location is to be determined.

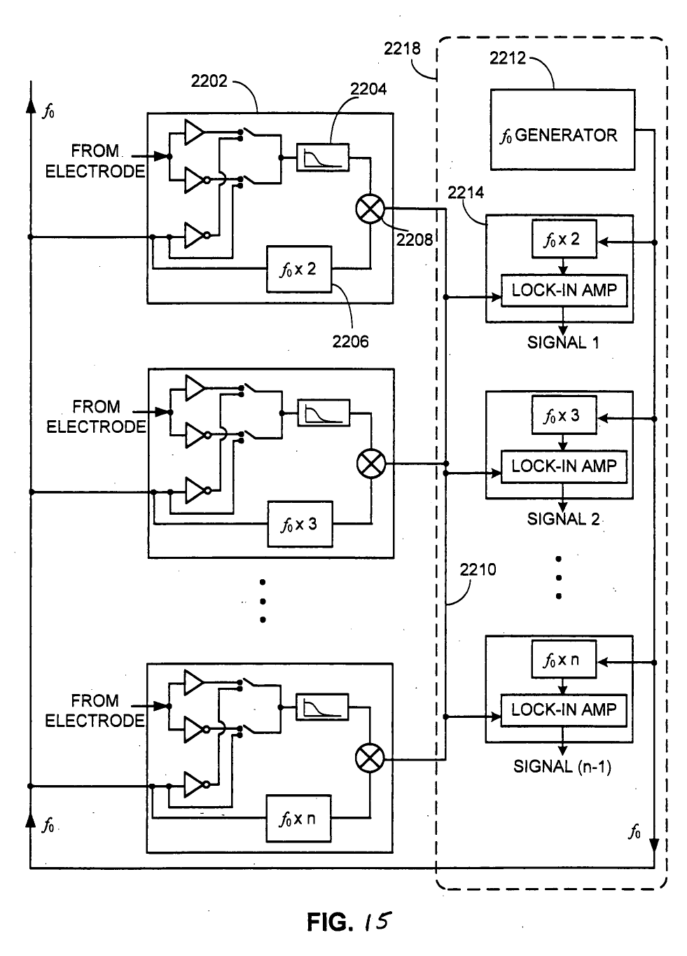

FIG. 15 illustrates one embodiment of a system of the present invention that enables simultaneous transmission of tomography signals over a single wire using frequency division multiplexing. During operation, the system applies an AC voltage with a base frequency f₀ across the tissue region. Every electrode is equipped with a multiplexer module, such as module 2202. A module has two inputs: one from the electrode for the tomography signal, and one for the base frequency f₀.

For example, in module 2202, the tomography signal is first amplified and then multiplied with the base frequency f₀. Note that in the example shown in FIG. 15, module 2202 also facilitates two switches, which enable an arbitrary selection of the sign for the tomography signal and the base-frequency signal. A low-pass filter 2204 then filters the multiplied signal. The cut-off frequency of low-pass filter 2204 is approximately the same as the base frequency f₀ (e.g., 100 KHz). Therefore, low-pass filter 2204 can use a capacitor with a more compact size, which allows module 2202 to reside locally with the electrode.

Meanwhile, a frequency multiplier 2206 multiplies the base frequency and produces a carrier frequency 2f₀, which is specific to module 2202. A frequency mixer 2208 subsequently mixes the filtered signal with the carrier frequency, and transmits the output signal to a common signal-return wire 2210.

Within each frequency-division-multiplexer module, the frequency multiplier multiplies the base frequency with a different factor. Consequently, the tomography signal from every electrode is carried by a different carrier frequency, i.e., 2f₀, 3f₀, . . . , nf₀. The system can therefore simultaneously transmit multiple tomography signals over a signal wire with minimum cross talk between the signals.

The demultiplexer circuits may reside in an external system 2218 or in a pacing can. For each tomography signal, there is a demultiplexer module, such as demultiplexer module 2214. Within a demultiplexer module is a frequency multiplier that produces a carrier frequency same as the carrier frequency for a tomography signal, using the same base frequency f₀. Also included in a demultiplexer module is a conventional lock-in amplifier operating at the carrier frequency supplied by the frequency multiplier. In this way, the system can demultiplex the mixed signals at different carrier frequencies and reproduce each tomography signal. In addition, demultiplexing system 2218 may also include a base-frequency generator 2212 that provides the f₀ signal to the demultiplexer modules as well as the multiplexer modules.

Embodiments of the subject systems incorporate other physiologic sensors in order to improve the clinical utility of wall-motion data provided by the present invention. For example, an integrated pressure sensor could provide a self-optimizing cardiac resynchronization pacing system with an important verification means, since wall motion optimization in the face of declining systemic pressure would be an indication of improper pacing, component failure or other underlying physiologically deleterious condition (e.g., hemorrhagic shock). One or more pressure sensors could also provide important information used in the diagnosis of malignant arrhythmias requiring electrical intervention (e.g., ventricular fibrillation). Incorporation of other sensors is also envisioned.

Effectors of interest include, but are not limited to, those effectors described in the following applications by at least some of the inventors of the present application: U.S. patent application Ser. No. 10/734490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No.10/764,125, entitled “Method and System for Remote Hemodynamic Monitoring”; International Application No. PCT/ US2005/046815 titled: “Implantable Hermetically Sealed Structures”; U.S. application Ser. No. 11/368,259 titled: “Fiberoptic Tissue Motion Sensor”; International application Ser. No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

In the implantable embodiments of this invention, as desired wall motion, pressure and other physiologic data can be recorded by an implantable computer. Such data can be periodically uploaded to computer systems and computer networks, including the Internet, for automated or manual analysis.

Uplink and downlink telemetry capabilities may be provided in a given implantable system to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., intracardiac electrocardiogram amplitude values, and sensor output signals including dimension signals developed in accordance with the invention. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

Data Processing

The electrical tomography data obtained using electrical tomography methods and systems, e.g., as described above, may be employed raw or processed as desired, e.g., depending on the particular application which the data is being employed.

In certain embodiments, the data is employed, either alone or in combination with non-ET data (such as data obtained from other types of physiological sensors, e.g., pH sensors, pressure sensors, temperature sensors, etc.) to determine one or more physiological parameters of interest, such as cardiac parameters of interest.

Parameters of cardiac performance measured using this approach can be measured both directly and indirectly. Examples of parameters which can be directly measured include, but are not limited to: cardiac wall motion, including measurements of both intra-ventricular and inter-ventricular synchrony; measurements of myocardial position, velocity, and acceleration in both systole and diastole; measurements of mitral annular position, velocity, and acceleration in both systole and diastole, including peak systolic mitral annular velocity; left ventricular end-diastolic volume and diameter; left ventricular end-systolic volume and diameter; ejection fraction; stroke volume; cardiac output; strain rate; inter-electrode distances; beat-to-beat variation; and QRS duration . Parameters which can be measured indirectly include, but are not limited to: dP/dt (a proxy for contractility); dP/dtₘₐₓ;; and calculated measurements of flow including mitral valve flow; mitral regurgitation; stroke volume; , and cardiac output. Other parameters which can be measured using the inventive electrical tomography system which are helpful in management of cardiac patients include, but are not limited to: transthoracic impedance, cardiac capture threshold, phrenic nerve capture threshold, temperature, respiratory rate, activity level, hematocrit, heart sounds, sleep apnea determination. In some embodiments, addition sensors (e.g. flow sensors, temperature sensors, pressure sensors, accelerometers, microphone, etc.) may be used to obtain physiologic or cardiac parameters. Both the raw data obtained with this method and processed data can be displayed and used to evaluate cardiac performance.

Parameters which can be measured using the inventive ET system or used in conjuction with ET system data include but are not limited to the following:

| Name | Variable | How Measured | Description of Utility |
|---|---|---|---|
| Contractility | dP/dt | Indirect | Change in left ventricular |
| | | Calculate systolic velocity of mitral | pressure over change in |
| | | annulus which correlates with dP/dt | time, Used as a proxy for |
| | | | contractility. |
| | dP/dt_{Max} | Calculate max systolic velocity of mitral | |
| | | annulus which correlates with dP/dtₘₐₓ | |
| Rate of decline in LV | −dP/dt_{Max} | Indirect (see above) | Used as a proxy for |
| pressure in early diastole | | | contractility |
| End diastolic pressure | EDP | Direct | Gauge Pressure in |
| | | Designated sensor; or | chamberwhen volume is |
| | | Indirect | maximum |
| | | Measure valve area using ET data, then | |
| | | use formula: | |
| | | ${( \frac{value area}{0.11 * SV} )}^{2} = Δ P$ | |
| | | Add ΔP to peripheral diastolic pressure to | |
| | | get ventricular diastolic pressure | |
| End systolic pressure | ESP | Direct | Gauge Pressure in chamber |
| | | Designated sensor; or | when volume is minimum |
| | | Indirect | |
| | | Measure valve area using ET data, then | |
| | | use formula: | |
| | | ${( \frac{value area}{0.11 * SV} )}^{2} = Δ P$ | |
| | | Add ΔP to peripheral systolic pressure to | |
| | | get ventricular systolic pressure | |
| Left Ventricular Pressure | LVP | Direct | Gauge pressure in Left |
| | | Designated sensor in LV; or | Ventricle |
| | | Indirect | |
| | | Measure valve area using ET data, then | |
| | | use formula: | |
| | | ${( \frac{value area}{0.11 * SV} )}^{2} = Δ P$ | |
| | | Add ΔP to peripheral systolic or diastolic | |
| | | pressure to get ventricular systolic or | |
| | | diatolic pressure | |
| Left Atrial Pressure | LAP | Direct measurement Designated sensor | Reflective of LV filling |
| | | in LA | pressures, which change |
| | | | based upon pump function |
| | | | and fluid status |
| Aortic Pressure | AOP | Direct measurement | Gauge Pressure in aorta just |
| | | | distal to Aortic Valve |
| Pressure Reserve | PR | d(LVESP) / d(LVEDP) | Marginal change in end |
| | | | systolic pressure due to a |
| | | | marginal change in end- |
| | | | diastolic pressure |
| Atrial and Ventricular | | Direct | Volume of cardiac chambers |
| Volumes | | | |
| Left Ventricular End- | LVEDV | Direct | Can track long-term evolution |
| Diastolic Volume | | | of chamber dilation and |
| | | | remodeling |
| Left Ventricular End-Systolic | LVESV | Direct | Can track long-term evolution |
| Volume | | | of chamber dilation and |
| | | | remodeling |
| Left Ventricular Volume | VR | d(LVESV) / d(LVEDV) | Marginal change in end |
| Reserve | | | systolic volume due to a |
| | | | marginal change in end- |
| | | | diastolic volume |
| Atrial and Ventricular | | Direct | Can track long-term evolution |
| Diameters | | | of chamber dilation and |
| | | | remodeling |
| Left ventricular End- | | Direct | Can track long-term evolution |
| Diastolic Diameter | | Use end-diastolic ET position data of | of chamber dilation and |
| | | electrodes circumscribing the LV (e.g. | remodeling |
| | | LV, CS around base of LV and RV apex) | |
| | | to define diameter of LV. | |
| Left Ventricular End-Systolic | | DirectUse end-systolic ET position data | Can track long-term |
| Diameter | | of electrodes circumscribing the LV (e.g. | evolution of chamber dilation |
| | | LV, CS around base of LV and RV apex) | and remodeling |
| | | to define diameter of LV. | |
| Ejection Fraction | EF | Direct | Most commonly used |
| | | (LVEDV-LVESV) / LVEDV | parameter to track LV |
| | | | systolic function. Describes |
| | | | the percentage of blood |
| | | | ejected from a chamber |
| | | | (usually LV) during a cycle. |
| Stroke volume | SV | Direct | Standard cardiac index. |
| | | LVEDV-LVESV | Changes with various |
| | | Corrected for mitral regurgitation if | treatments and pathological |
| | | present (see below) | states. Net amount of blood |
| | | | ejected into aorta in one |
| | | | cycle. |
| | | Indirect | |
| | | Measure VTI × CSA = velocity-time | |
| | | integral of flow at mitral annulus x cross | |
| | | sectional area at mitral annulus | |
| | | Corrected for mitral regurgitation if | |
| | | present (see below) | |
| Stroke Volume Index | SVI | SV/BSA | Stroke volume normalized by |
| | | | Body Surface Area |
| Stroke Reserve | SR | d(SV) / d(LVEDP) | Marginal increase in stroke |
| | | | volume due to a marginal |
| | | | increase in LVEDP |
| Stroke Reserve Index | SRI | d(SVI) / d(LVEDP) | Stroke Reserve normalized |
| | | | by Body Surface Area |
| Stroke Work | SW | $SV * \overline{{AOP}_{Systole}} - \overline{{LVP}_{Diastole}}$ | Hemodynamic work performed by the left ventricle during a single cycle |
| Stroke Work Index | SWI | SW/BSA | Stroke Work normalized by |
| | | | Body Surface Area |
| Stroke Work Reserve | SWR | d(SW) / d(LVEDP) | Marginal increase in Stroke |
| | | | Work due to a marginal |
| | | | increase in LVEDP |
| Stroke Work Reserve Index | SWRI | SWR/BSA | Stroke Work Reserve |
| | | | normalized by Body Surface |
| | | | Area |
| Stroke Power | SP | SW / SEP | Power performed by heart |
| | | | against circulatory system |
| Stroke Power Index | SPI | SP / BSA | Stroke Power normalized by |
| | | | Body Surface Area |
| Stroke Power Reserve | SPR | d(SP) / d(LVEDP) | Marginal increase Stroke |
| | | | Power due to a marginal |
| | | | increase in LVEDP |
| Stroke Power Reserve | SPRI | SPR / BSA | Stroke Power Reserve |
| Index | | | normalized by body surface |
| | | | areas |
| Cardiac output | CO | Direct or Indirect, depending on method | Very commonly used cardiac |
| | | of measuring SV (see above) | index. Derivative of SV. |
| | | SV X HR | Total amount of blood |
| | | (stroke volume × heart rate) | pumped by the heart per |
| | | | minute. |
| Cardiac Index | CI | CO/BSA | Cardiac output normalized by |
| | | | Body Surface Area |
| Cardiac Reserve | CR | d(CO) / d(LVEDP) | Marginal increase in cardiac |
| | | | output due to a marginal |
| | | | increase in LVEDP |
| Cardiac Reserve Index | CRI | d(CI) / d(LVEDP) | Cardiac Reserve normalized |
| | | | by Body Surface Area |
| Myocardial Work | MyW | ${\int }_{dVldt < 0} Pdv - {\int }_{dVldt > 0} Pdv$ | Work performed by myocardial tissue during a single cycle |
| Myocardial Work Moment | MyWM | ${\int }_{dVldt < 0} PVdv - {\int }_{dVldt > 0} PVdv$ | Work moment performed by myocardial tissue during a single cycle |
| Myocardial Work Index | MyWI | MW / BSA | Myocardial work normalized |
| | | | by Body Surface Area |
| Myocardial Reserve | M_{y}R | d(MW)/d(LVEDP) | Marginal increase in |
| | | | myocardial reserve due to a |
| | | | marginal increase in LVEDP |
| Myocardial Reserve Index | M_{y}RI | d(MWI)/d(LVEDP) | Myocardial Reserve |
| | | | normalized by Body Surface |
| | | | Area |
| Myocardial Power | MyP | MyW / SEP | Power performed by the |
| | | | myocardia during systole |
| Myocardial Power Index | MyPI | MyP / BSA | Myocardial Power normalized |
| | | | by body surface area |
| Myocardial Power Reserve | MyPR | d(MyP) / d(LVEDP) | Marginal increase in |
| | | | myocardial power due to a |
| | | | marginal increase in end |
| | | | diastolic pressure |
| Myocardial Power Reserve | MyPRI | MyPR / BSA | Myocardial Power reserve |
| Index | | | normalized by body surface |
| | | | area |
| Myocardial Power | MyPSV | MyP / SV | Power required to deliver unit |
| Requirement | | | stroke volume |
| Cardiac Efficiency | CE | SW/ M_{y}W | Efficiency of the heart in |
| | | | converting myocardial work |
| | | | into circulatory work |
| Cardiac Amplification | CA | d(SV) / d(LVEDV) | Marginal increase in stroke |
| | | | volume due to a marginal |
| | | | increase in LVEDV |
| ET systolic measurements | Sₘ-ET, | Direct | To detect regional wall |
| | e.g. | Measure systolic displacement, velocity, | motion abnormalities, a |
| | | and acceleration data from ET sensing | hallmark of prior infarct (if |
| | | electrodes (e.g., “Sₘ-Et” would be the ET | unchanged over time) or |
| | | correlate of Sₘ, the maximal velocity of a | ischemia (if dynamically |
| | | segment of myocardium as measured by | changing over time) |
| | | TDI) | |
| ET diastolic measurements | Eₐ-ET, e.g. | Direct | Can help to diagnose, |
| | | Measure diastolic displacement, velocity, | differentiate and follow |
| | | and acceleration data from ET sensing | various forms of diastolic |
| | | electrodes. | dysfunction |
| | | “Eₐ-ET” is the ET correlate of Eₐ, the | |
| | | maximal velocity of the MV annulus | |
| | | during early diastolic filling, as measured | |
| | | by TDI) Can be measured from MV | |
| | | annulus (CS) or from other parts of the | |
| | | myocardium. | |
| ET diastolic measurements | | Direct | Can help to diagnose, |
| of LV diastolic filling | | Measured at mitral annulus or left | differentiate and follow |
| Left Ventricular Inflow | | ventricular free wall. | various forms of diastolic |
| Velocities | | | dysfunction. Many of these |
| | | | parameters are standard |
| | | | components of an |
| Early Diastolic Filling | E | Measure early maximum filling velocity of | examination for diastolic |
| Velocity | | ventricle after opening of mitral valve. | dysfunction. |
| Filling Velocity after Atrial | A | Measure second velocity peak in late | |
| Contraction | | diastolic period after atrial contraction. | |
| Ratio of Early Diastolic | E/A | Ratio of E/A | |
| Filling Velocity to Filling | | | |
| Velocity after Atrial | | | |
| Contraction | | | |
| Acceleration/Deceleration | | | |
| Maximal acceleration | | Measure acceleration (derivative of | |
| | | velocity) from time of onset to flow to E | |
| | | velocity. | |
| Early diastolic deceleration | | Measure deceleration from E velocity | |
| slope | | peak to zero baseline. | |
| Myocardial Tissue Velocities | | | |
| Early diastolic myocardial | Eₘ | Measure velocity of myocardium in early | |
| tissue velocity | | diastole. | |
| Diastolic myocardial tissue | Aₘ | Measure velocity of myocardium in after | |
| velocity after atrial | | atrial contraction. | |
| contraction | | | |
| Ratio of early diastolic | Eₘ/ Aₘ | Ratio of Eₘ / Aₘ | |
| myocardial | | | |
| tissue velocity and diastolic | | | |
| myocardial tissue velocity | | | |
| after atrial contraction | | | |
| A wave velocity | | Direct | Can help to diagnose, |
| | | Measure small reversal of flow in atrium | differentiate and follow |
| | | following atrial contraction (a wave) | various forms of diastolic |
| | | | dysfunction. |
| E wave velocity | | Direct | Can help to diagnose, |
| | | Measure small reversal of flow at end- | differentiate and follow |
| | | systole (v wave) | various forms of diastolic |
| | | | dysfunction. |
| Propagation Velocity | | Indirect | Measures velocity as blood |
| | | May be estimated from velocity at mitral | moves from mitral annulus to |
| | | annulus, LV free wall, or septal electrode | LV apex |
| Cardiac wall motion | | Direct | To detect regional wall |
| | | Measure ET motion (displacement, | motion abnormalities, a |
| | | velocity, acceleration) data from | hallmark of prior infarct (if |
| | | electrodes on cardiac wall. | unchanged over time) or |
| | | | ischemia (if dynamically |
| | | | changing over time) |
| Intraventricular synchrony | | Direct | Predictor of CRT response; |
| | | Compare timing of ET motion data from | assessment of CRT |
| | | various electrodes around LV. | response |
| Interventricular synchrony | | Direct | Predictor of CRT response; |
| | | Compare timing of ET motion data from | assessment of CRT |
| | | electrodes in LV to pressure | response |
| | | measurement in RV, and / or to timing of | |
| | | electrodes in the RV. | |
| Myocardial position, | | Direct | To detect regional wall |
| velocity, acceleration | | Measure ET motion (position, velocity, | motion abnormalities, a |
| | | acceleration) data from electrodes on | hallmark of prior infarct (if |
| | | cardiac wall, in both systole and diastole. | unchanged over time) or |
| | | | ischemia (if dynamically |
| | | | changing over time) |
| Mitral annular position, | | Direct | Provides important systolic |
| velocity, acceleration | | Calculate velocity and acceleration from | and diastolic data. |
| | | ET position data of electrodes in coronary | Systolic velocity correlates |
| Peak Systolic Mitral Annular | | sinus (CS) wrapping around mitral | with dP/dtₘₐₓ |
| Velocity | | annulus, in both systole and diastole. | |
| Mitral valve flow | | | MR can change with |
| | | Indirect | pharmacologic and device- |
| | | Measure VTI × CSA = velocity-time | based interventions (e.g., |
| | | integral of flow at mitral annulus × cross | CRT). |
| | | sectional area at mitral annulus | |
| Mitral regurgitation | MR | Indirect | MR can respond to |
| | | Measure VTI × CSA = velocity-time | pharmacologic and device- |
| | | integral of retrograde flow at mitral | based interventions (e.g., |
| | | annulus × cross sectional area at mitral | CRT). MR can also come |
| | | annulus | and go with ischemia in some |
| | | | patients |
| | | Indirect | |
| | | Measure cross-sectional area of mitral | |
| | | annulus to infer degree of closure of the | |
| | | leaflets. | |
| Valvular Gradient | VG | ΔPmax | Maximum (during a cycle) |
| | | | pressure gradient across a |
| | | | valve |
| Valvular Gradient Reserve | VGR | d(VG) / d(LVEDP) | Increase in VG as a function |
| | | | of increase in LVEDP. |
| Valvular Area | VA | $0.11 * SV \sqrt{Δ P}$ | Standard calculation of valvular area using mean pressure gradient and mean flow rate |
| Valvular Area Reserve | VAR | d(VA) / d(LVEDP) | Increase in valvular area as a |
| | | | function of increase in |
| | | | LVEDP |
| Valvular Regurgitation | VR | $\int {Q}_{REGURGITATION}$ | Cumulative regurgitant flow during a cycle |
| Valvular Regurgitation | VRR | d(VR) / d(LVEDP) | Increase in regurgitant flow |
| Reserve | | | as a function of increase in |
| | | | LVEDP |
| Filling Rates in Atrium and | | Direct | Can help to diagnose, |
| Ventricle | | | differentiate and follow |
| | | | various forms of diastolic |
| Peak Rapid Filling Rate | | Measure peak velocity and acceleration | dysfunction. |
| | | of ET signals for S, E, and A waves | |
| Peak Atrial Filling Rate | | | |
| Fractional Filling Rates | | | |
| Interelectrode distances | | Direct | Measures strain rate, a |
| | | Calculate change in distance of | predictor of CRT response. |
| | | electrodes within close proximity using | |
| | | ET motion data. | |
| Left Ventricular Twist Index | | Direct | Measures degree of |
| | | Measure angular component of velocity | ventricular twisting of apex |
| | | of free wall electrode(s) | with respect to the base |
| Myocardial Strain | | Direct | Predictor of CRT response |
| | | Calculate change in distance of | |
| | | electrodes within close proximity using | |
| | | ET motion data. | |
| Myocardial Strain Rate | SR | Direct | Predictor of CRT response. |
| Max Myocardial Strain Rate | SRₘₐₓ | Calculate rate of change in distance of | |
| | | electrodes within close proximity using | |
| | | ET motion data. | |
| Diastolic Time Intervals | | Direct | Can help to diagnose, |
| | | | differentiate and follow |
| Isovolumetric Relaxation | IVRT | Time between aortic valve closure and | various forms of diastolic |
| Time | | the onset of ventricular filling (mitral valve | dysfunction. |
| | | opening) | |
| Deceleration Time | DT | Time between Epeak velocity and zero | |
| | | baseline | |
| Atrial Filling Period | A_{dur} | Measured at mitral annulus | |
| Time from mitral valve | | Time from mitral valve opening to early | |
| opening to E velocity | | maximum diastolic filling velocity | |
| Systolic Time Intervals | | Direct | Can help to diagnose, |
| | | differentiate and follow | |
| Systolic Ejection Period | SEP | Time during which blood is ejected from | various forms of systolic |
| | | LV into Aorta | dysfunction, including |
| | | | evaluation of systolic |
| Time to Onset of Systolic | | Time from beginning of QRS complex to | dyssynchrony. |
| Velocity | | beginning of S wave | |
| Time to Peak Systolic | Ts | Time from beginning of QRS complex to | |
| Velocity | | peak of S wave. | |
| Time to peak acceleration | | Time to maximum systolic acceleration | |
| | | Time from beginning of QRS complex to | |
| Time to Peak Post-Systolic | | peak post-systolic velocity. | |
| Velocity | | | |
| | | Time from beginning of QRS complex to | |
| | | maximum systolic displacement. | |
| Time to Maximal Systolic | Td | | |
| Displacememt | | | |
| Beat to beat variation | R-R | Direct | Can get early warning of |
| | interval | Measure variability of R-R period from | decompensation in heart |
| | | ECG measurements or from ET electrode | failure (HF) and coronary |
| | | motion. | artery disease (CAD) |
| | | | patients. |
| Valve Timing | | Direct | |
| | | Measure impedance change as valve | |
| | | opens | |
| QRS Duration | QRS | Direct | |
| | | Measure length of QRS interval from | |
| | | ECG measurements or from ET electrode | |
| | | motion. | |
| Transthoracic Impedance | | Direct | Can get early warning of |
| | | Thoracic impedance correlates with fluid | decompensation in heart |
| | | status. | failure. |
| Cardiac Capture Threshold | | Direct | To determine lowest |
| | | Measure from EKG or ET electrodes | threshold where cardiac |
| | | | stimulation can be achieved. |
| Heart sounds | | Direct | Measures the timing of |
| | | Microphone or accelerometer in | opening and closing of |
| | | implantable pulse generator. | valves. Helps clarify timing |
| | | | of events in the cardiac cycle. |
| Phrenic Nerve Capture | | Direct | Detect in order to avoid |
| Threshold | | Manifests as sharp spike in ET position | unwanted diaphragmatic |
| | | data. | stimulation |
| Temperature | | Direct | |
| | | Thermocoupling | |
| Respiratory rate | RR | Direct | |
| | | Can detect from impedance data, or | |
| | | signal from ET data | |
| Activity level | | Indirect | |
| | | Accelerometer in implantable pulse | |
| | | generator | |
| Hematocrit | HCT | Direct | |
| | | Blood resistiviy | |

As such, a value for a parameter of interest can be obtained from the ET data provided by the methods and systems. The parameter can be one that is derived solely from ET data, or one that is derived from both ET and non-ET data, e.g., data from other types of physiological sensors, e.g., as described above.

Displaying Data

In certain embodiments, the obtained data is displayed to a user, where the displayed data may be raw data or data that has been processed, e.g., using one or more data processing algorithms. The displayed data may be displayed in any convenient format, e.g., printed onto a substrate, such as paper, provided on a display of a computer monitor, etc. The displays may be in the form of plots,. graphs, or any other convenient format, where the formats may be two dimensional, three-dimensional, included data from non-ET sources, etc. Displays of interest include, but are not limited to: those disclosed in PCT application serial no. PCT/US2006/012246 titled “Automated Optimization of Multi-Electrode Pacing for Cardiac Resynchronization,” and filed on Mar. 31, 2006, the disclosure of which is herein incorporated by reference.

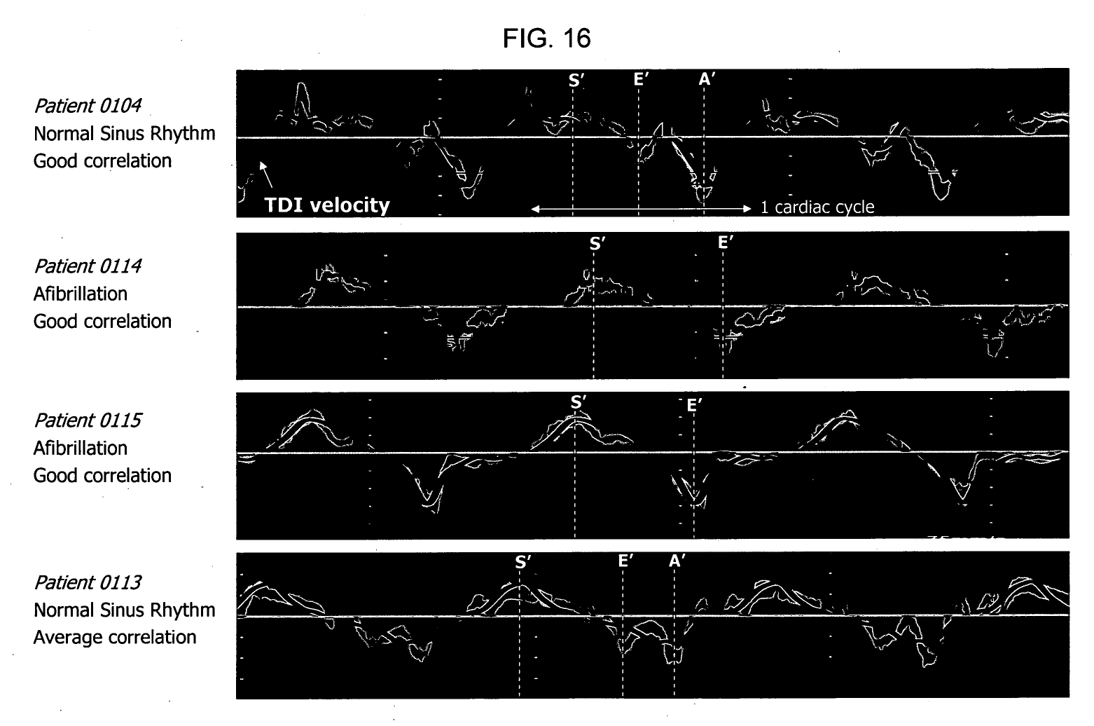

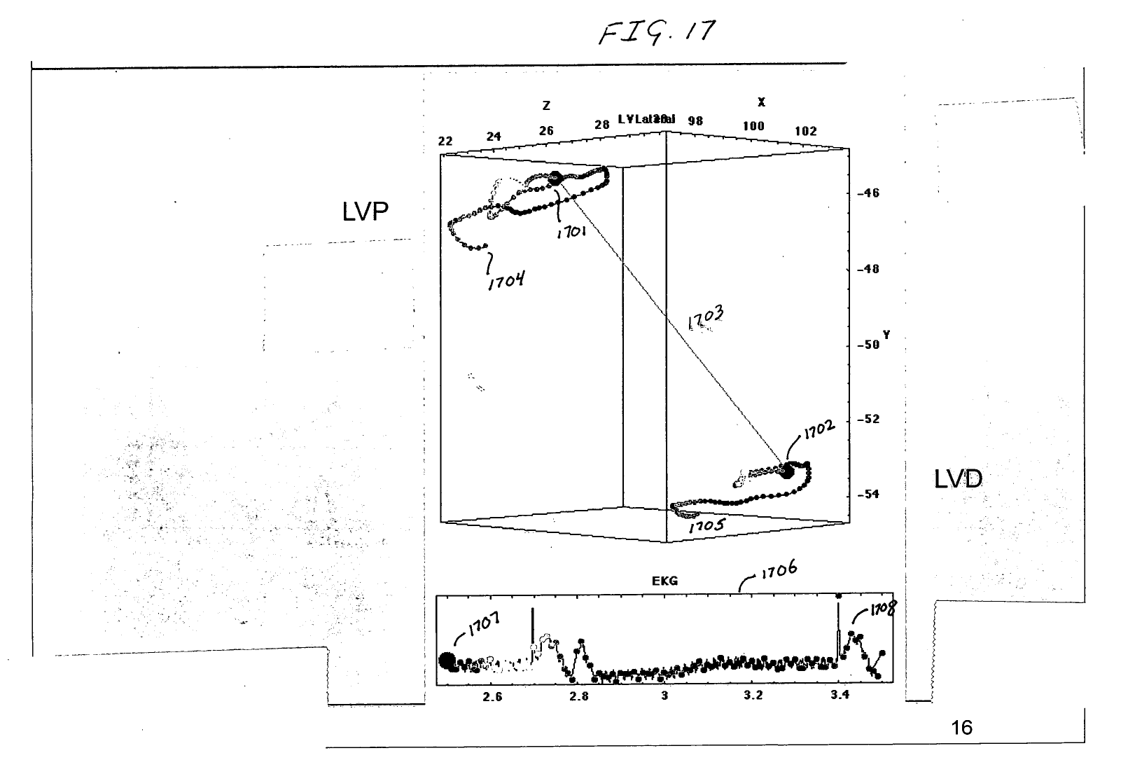

As such, the ET data obtained from electrical tomography methods and systems of the invention, e.g., as described above, can be processed and displayed in a number of ways useful to clinicians treating patients, e.g., patients who are undergoing CRT. In certain embodiments, the motion of one or more sense electrodes (e.g. one or more sense electrodes on the same cardiac lead, or one or more sense electrodes on different cardiac leads) can be evaluated in one- or two-dimensional space, by producing a position plot, such that a one- or two-dimensional display of the position plot of the sense electrode(s) as it changes over time is provided. For example, a one-dimensional plot of position (i.e. a linear plot) in the X, Y, or Z plane as a function of time can be displayed. In another embodiment, a two-dimensional plot of position as a function of time can be displayed, e.g. in the XZ or XY plane. In certain embodiments, the motion of one or more sense electrodes (e.g. one or more sense electrodes on the same cardiac lead, or one or more sense electrodes on different cardiac leads) can be evaluated in three-dimensional space, by producing a position plot, such that a three-dimensional display of the position plot of the sense electrode(s) as it changes over time is provided, as shown in FIG. 17. FIG. 17 illustrates one embodiment of a three-dimensional display of position as a function of time, where two left ventricular sense electrodes on the same lead are shown. In this illustration, the proximal electrode, i.e. the electrode closest to the implantable pulse generator, is labeled “LVP” (1701 in FIG. 17). The distal electrode, i.e. the electrode farthest from the implantable pulse generator, is labeled “LVD” (1702 in FIG. 17). The lead is depicted by a line connecting the LVP and LVD electrodes (1703). The three-dimensional display of position as a function of time for one or more electrodes can be shown as a tracing of the path of the electrode (1704 for the LVP electrode, and 1705 for the LVD electrode) which shows the motion of each sense electrode over a period of time (e.g. the duration of one or more cardiac cycles). The three-dimensional display of position as a function of time for one or more electrodes can be animated, which allows visualization of the motion at each sense electrode in real-time. Where desired, the three-dimensional plot of motion can be displayed with one or more additional plots, such as one or more additional plots of parameters of cardiac function, including plots derived from non-ET obtained data, such as a simultaneously obtained electrocardiogram (EKG), for example 1706 in FIG. 17, a plot of TDI velocity shown with ET-derived data (as shown in FIG. 16), etc.

In certain embodiments, the two or more distinct plots in which one of the plots is derived from ET data, such as a three-dimensional plot of motion, can have color coded data points. The plot can be labeled such that a single point in the cardiac cycle (e.g. the beginning of ventricular systole) is labeled with the same color in each of the two or more plots. The color of each labeled point can be unique for each time point in the cardiac cycle. For example, in FIG. 17, the color red is used to mark the initial path of the motion of the electrodes (1704 and 1705), as well as the initial tracing on the EKG (1707), which correspond to the same point in time. The display can also include a colored dot which moves along the path of the electrode in a three-dimensional plot (1701 and 1702), and along the tracing of an additional plot (such as an EKG) (1708), and marks the same time point with respect to the cardiac cycle in each of the multiple plots. Additionally, the time point corresponding to the beginning of each R wave in the cardiac cycle can be identified by a color discontinuity. The display can further include a plot of respiratory signal which is also color labeled, and can be incorporated into any of the display modes and features as described above.

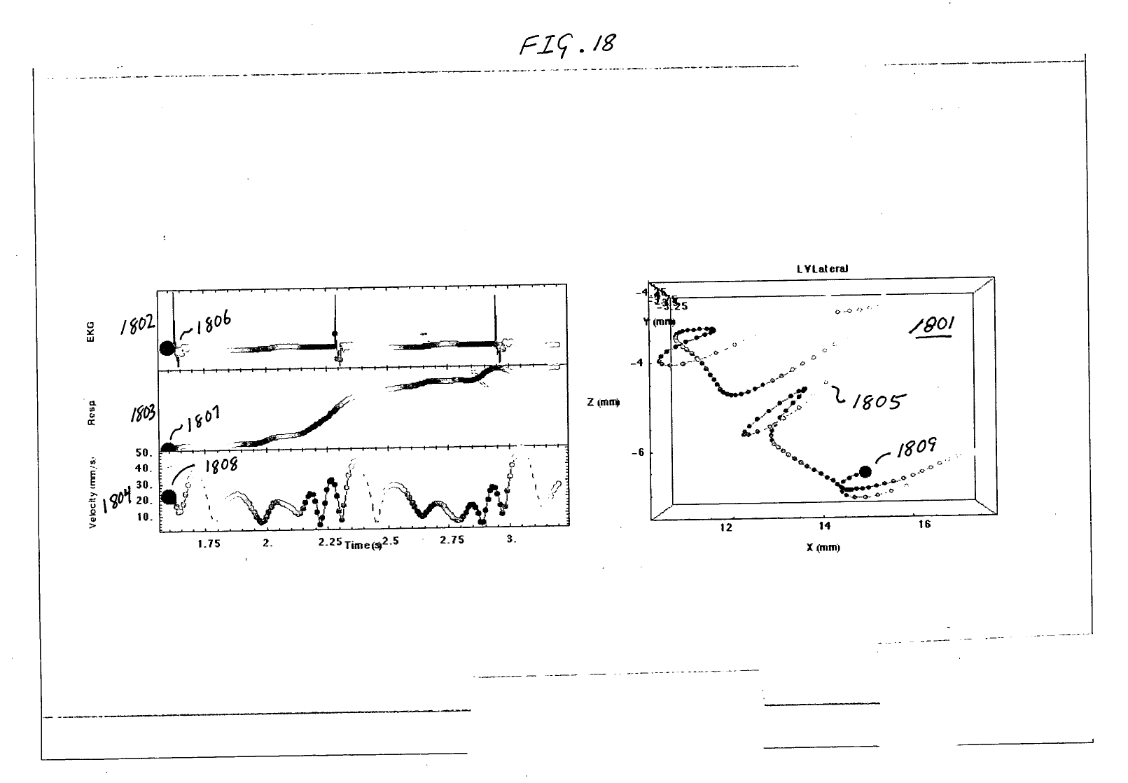

A three-dimensional display of motion at one or more sense electrodes (1801 in FIG. 18), along with a simultaneously obtained additional plot(s), such as EKG (1802), respiratory signal (1803), or velocity plot (1804), as shown in FIG. 18, can be used, for example, in the evaluation of patients undergoing cardiac resynchronization therapy (CRT). A three-dimensional display can provide a useful and easily understandable method of demonstrating heart motion and function to a clinician (e.g. in comparing cardiac motion at baseline, and with CRT turned on). Additional parameters calculated from the motion of the electrodes as well as comparison of motion between electrodes also provide valuable information about heart motion and function which is useful for CRT. As described in FIG. 17, a three-dimensional display of position as a function of time for one or more electrodes can be shown as a tracing of the path of the electrode (1805 in FIG. 18) By integrating over the area circumscribed by the plot of motion during the cardiac cycle (1805), for example, one can obtain a measure of total motion, and by maximizing the area circumscribed by the plot of one or more electrodes during the cardiac cycle, one can obtain an objective measurement of cardiac motion useful for optimizing CRT.

In certain embodiments, the display can also include a colored dot which moves along the path of the electrode in a three-dimensional plot (1809), and along the tracing of the additional plot(s) such as an EKG (1806), a respiratory signal (1807), and a plot of total velocity (1808), for example, and marks the same time point with respect to the cardiac cycle in each of the multiple plots. In some embodiments, total velocity can be computed, which is the magnitude of the sum of the velocities of the electrode in all directions of motion, and as such is a positive number (1808).

In certain embodiments, a standard error, or standard deviation measurement, can be calculated for the position signal of an electrode calculated from monitored voltage data. The standard error, or standard deviation, can be useful as a measure of how widely spread the values are in a data set. The standard error or standard deviation can also be calculated for other measurements derived from the position data, such as velocity, or acceleration. The standard error can be calculated for any measurement or plot of position, velocity, or acceleration where desired. The standard error measurement can also be included as part of the display of a one-, two-, or three-dimensional plot of position, velocity, or acceleration where desired; e.g. as standard error bars.

The three-dimensional display can be oriented so that the three essentially orthogonal fields defining the display are aligned with the principle axes of the heart, such that a first plane or axis is parallel to the long axis of the left ventricle (“long-axis plane”), a second plane is oriented perpendicular to the first (“short-axis plane”), and a third plane is perpendicular to both the long- and short-axis planes (“four-chamber plane”). This orientation of the three-dimensional display can correlate with the standard views of the heart typically obtained with echocardiography.

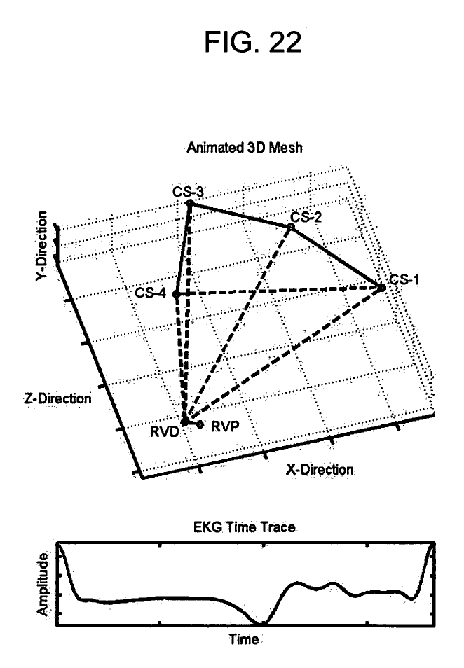

Another embodiment of a three-dimensional display for use with the subject methods is a method for evaluating a three-dimensional volume bounded by four or more electrodes, e.g. as shown in FIG. 22. FIG. 22 illustrates a three-dimensional matrix created by four electrodes around the coronary sinus (CS-1, CS-2, CS-3, CS-4) and a right ventricular distal electrode (RVD). The proximal right ventricular electrode is also shown (RVP). As the right ventricular distal electrode is in close proximity to the left ventricular apex, calculating the volume outlined by the coronary sinus electrodes at the base of the heart and the RVD electrode at the apex can provide a measurement of left ventricular volume. Similar methods employing the same or other electrodes can be used to measure the volumes of other cardiac chambers of interest. By detecting changes in volumes or distances defined by some or all of the electrodes, a variety of different cardiac function parameter may be determined.

In certain embodiments, the data is displayed to a user in a graphical user interface. The phrase “graphical user interface” (GUI) is used to refer to a software interface designed to standardize and simplify the use of computer programs, as by using a mouse to manipulate text and images on a display screen featuring icons, windows, and menus. GUIs of interest include, but are not limited to: those disclosed in PCT application serial no. PCT/US2006/012246 titled “Automated Optimization of Multi-Electrode Pacing for Cardiac Resynchronization,” and filed on Mar. 31, 2006, the disclosure of which is herein incorporated by reference. GUI displays can be tailored to assist the clinician during clinical situations, such as but not limited to: during implantation of the sensing or pacemaker leads; during initial adjustment of CRT parameters or later “tune-up” of CRT parameters in the clinician's office; and for long-term tracking of cardiac performance.

During implantation of the sensing or pacemaker leads, three-dimensional motion-tracking software can be used to generate a motion-tracking position plot of the motion of a first sense electrode (e.g. a right ventricular electrode on a lead or guidewire) during placement of the electrode (e.g. in the right ventricle). The motion-tracking position plot of the sense electrode can be plotted in three-dimensional space, and can be displayed together with, and aligned with, a fluoroscopic image in the graphical user interface. The motion-tracking .position. plot of the sense electrode can be dynamically displayed, meaning that the three-dimensional display can change in real-time (e.g. with motion of the heart at different points in the cardiac cycle, with motion of the patient, with motion of the electrode and/or the lead, etc.) and/or with changes in the desired angle of view, or projection of the display. The fluoroscopy images can be evaluated with motion tracking software to obtain fluoroscopy motion data. The fluoroscopy image can be calibrated using the known electrode sizes, and then both the fluoroscopy and ET data can be displayed together, with offset as needed for the best matching of the images. The motion-tracking position plot of the electrode and the fluoroscopic image can be constantly updated based on motion or changes in position or view, and can be rotationally displayed in three-dimensional space, meaning that the display of both the motion-tracking position plot and the fluoroscopic image can be rotated together, around any axis, in any direction where desired for optimal viewing. The system can also retain the information from each attempted location or placement of a lead or guidewire to create an anatomic map of the heart. If a clinician wants to return to a position or placement previously attempted, the data can be stored and available for retrieval during the implantation. An additional feature of the ET data display system is the optional automatic calculation of pacing intervals to test during implantation of a lead. For example, a clinician may want to automatically test cardiac pacing parameters at different distances, e.g., every 5 mm, or every 10 mm, as a lead or guidewire is advanced in a coronary vein. Using the ET system, the clinician can be notified every time the desired distance between pacing locations is reached, and cardiac pacing can be performed automatically at that site.

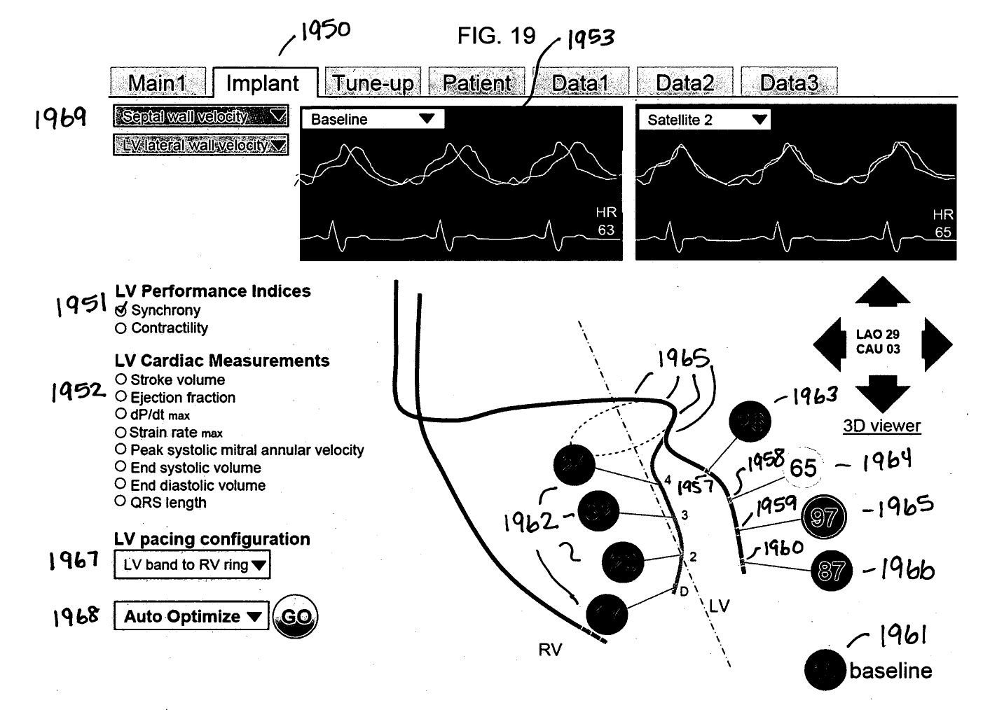

Motion-tracking of at least a second lead can also be displayed (e.g. a left ventricular electrode on a lead or guidewire) and motion tracking software can include a tool to assist in the technically difficult location of the opening of the coronary sinus. The three-dimensional display can include a feature that allows the clinician to mark locations where there has been an unsuccessful attempt to locate the coronary sinus (e.g. with a red dot), thereby avoiding additional unsuccessful attempts in the same location. In addition, with ET data, an unlimited number of ‘views’ of the heart can be displayed, because each ‘view’ is a mathematical computation. ET can therefore not only create views similar to the standard fluoroscopic views, but can also display data in projections that would not be possible with fluoroscopy alone. For example, during an attempt to locate the coronary sinus, the preferred LAO (left anterior oblique), RAO (right anterior oblique) and AP (anteroposterior) views used with standard fluoroscopy can not only be created and displayed simultaneously using ET data, but additional views, e.g. a cranial view, which cannot be provided with fluoroscopy, can also be displayed. The capacity of ET data to provide the clinician with multiple views simultaneously provides depth perception which is not possible with a single fluoroscopic view. These features available with ET data can decrease the time needed for successful placement of the lead, thereby reducing the fluoroscopy time and radiation exposure. In certain embodiments, the projection angle, or perspective, of one or more views of the heart can be shown on the screen, e.g. LAO or left anterior oblique view, 29 degrees, and CAU or caudal view, 3 degrees, as shown FIG. 19 (1970). In certain embodiments, one or more views of the heart can be displayed simultaneously on the screen. In some embodiments, the view can be changed or directed where desired by the clinician through the use of directional arrows on the screen (1970).

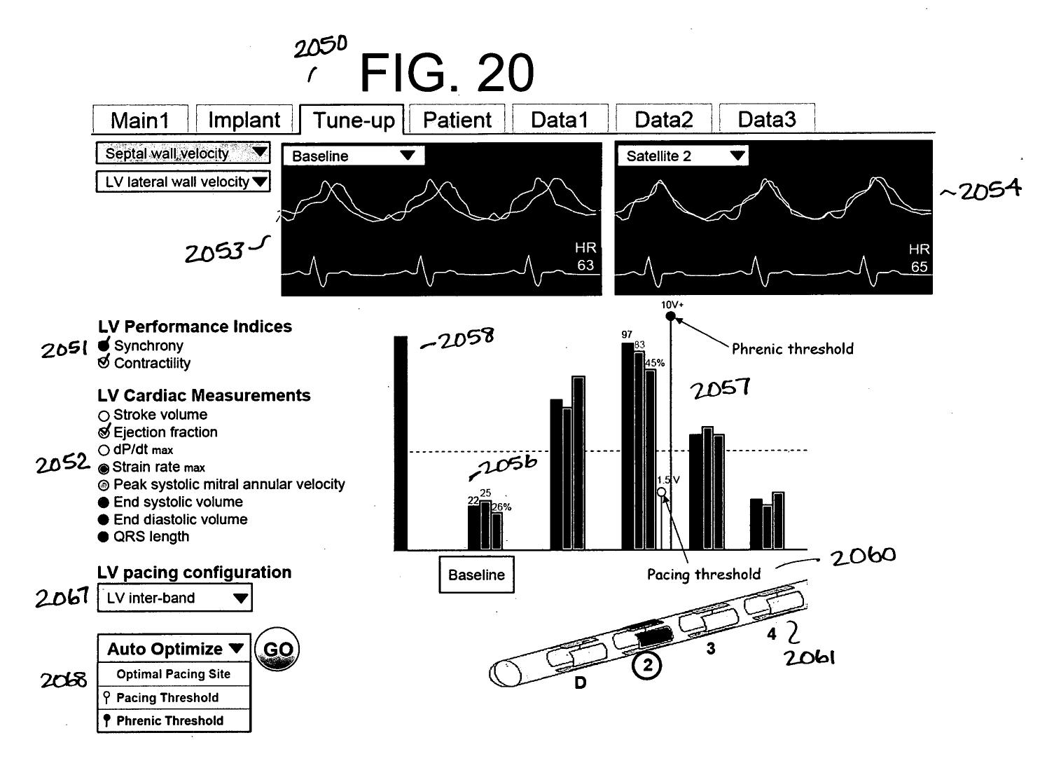

For the initial adjustment of CRT parameters at the time of implantation or later “tune-up” of CRT parameters in the clinician's office, in one embodiment, the desired display can be chosen from a menu bar at the top of the display screen depending on the task for be performed; for example “Implant” can be chosen when the initial adjustment of CRT parameters is to be performed (1950 in FIG. 19), or “Tune-up” can be chosen when CRT parameters are to be readjusted (2050 in FIG. 20). In some embodiments the display can include a normalized index of left ventricular performance (e.g. synchrony or contractility), (1951 in FIG. 19, 2051 in FIG. 20) or a left ventricular measurement which can be calculated or measured from one or more sense electrodes, e.g. on a lead (1952 in FIG. 19, 2052 in FIG. 20). LV performance indices displayed can include, but are not limited to, synchrony and contractility. LV cardiac measurements displayed can include, but are not limited to, stroke volume, cardiac output, ejection fraction, dP/dtₘₐₓ, strain rateₘₐₓ, peak systolic mitral annular velocity, end-systolic volume, end-diastolic volume, and QRS length. Where desired, the display can also include one or more additional plots, such as one or more additional plots of parameters of cardiac function, such as a graph of the degree of cardiac synchrony at baseline (1953 in FIG. 19, 2053 in FIG. 20), and the degree of cardiac synchrony when using a particular electrode for pacing (1954 in FIG. 19, 2054 in FIG. 20). In certain embodiments, the variables to be plotted on the graphs can be selected and/or changed by the clinician, e.g. septal wall velocity can be displayed as a function of time, or LV lateral wall velocity can be graphed as a function of time, as shown in FIG. 19 (1969; 2069 in FIG. 20). The display can also include one or more additional plots derived from non-ET obtained data, such as a simultaneously obtained electrocardiogram (EKG) (1955 in FIG. 19, 2055 in FIG. 20).

To generate the display, each sense electrode is employed for pacing in sequence, with the remaining electrodes on the lead or leads used for sensing. For example, in one embodiment a left ventricular lead can have eight electrodes; four motion-sensing electrodes located around the mitral annulus (1956), and four pacing and/or sensing electrodes located along the left ventricular free wall (1957,1958,1959, and 1960). The program software can automatically calculate left ventricular performance parameters when pacing is conducted from a first electrode (e.g. the most proximal electrode located along the left ventricular free wall, 1957), and after several beats switch to pacing from a second electrode on the lead (e.g. the second left ventricular electrode, 1958), while the first electrode reverts to a sensing function. The program software can then automatically calculate left ventricular performance parameters when pacing is conducted from the second electrode. The process can then be repeated for the third electrode on the lead, etc.

After all the potential pacing electrodes on the lead or leads have been tested as pacing electrodes, post-processing of the data generates a normalized index of LV performance or a LV cardiac measurement at baseline (1961), and for each electrode, based on the left ventricular performance parameters measured during pacing with that individual electrode (1962, 1963, 1964, 1965, 1966). A clinician can select the button for a particular parameter (e.g. “contractility” (1951) or “ejection fraction”(1952)), and the LV performance index or LV cardiac measurement of interest can be simultaneously displayed on the motion-tracking three-dimensional display of the cardiac lead as a number associated with each electrode. For example, in FIG. 19 the LV performance index that has been selected is “synchrony”. This number, or ‘score’ can be color-coded (for example, green for a good ‘score’; red for a suboptimal (‘score’). For example, in FIG. 19, the highest, or best, scores of “97” and “87” are displayed on a green background circle (1965, 1966). An intermediate score of “65” is displayed on a lighter green circle (1964). The lowest, or suboptimal scores are displayed on red background circles (1962, 1963). The clinician can then select the pacing electrode that has the most favorable index of LV performance or LV measurement as the pacing electrode. In the example of FIG. 19, the most favorable index is “97” (1965), generated by pacing with electrode 1959. If the results displayed are not optimal, the clinician can choose to reposition the lead (e.g. by moving the lead within the same coronary vein, or by selecting an alternate coronary vein) while the image of the previous vein and its indices of left ventricular performance remain on the screen. After repositioning of the electrodes, another pacing cycle with the electrodes in their new positions can be performed, until the desired result is achieved.

After an LV performance parameter of interest has been evaluated and an optimal pacing location is chosen, the pacing setting(s) can be optimized. Pacing settings that can be selected for manual or automated optimization include but are not limited to: pacing location, pacing electrode, stimulation strengths, and timing delay, as described below. The timing delay can be optimized, either by the clinician or with an auto-optimize cycle (1968 in FIG. 19, 2068 in FIG. 20) to optimize parameters including but not limited to the AV and W interval. In another embodiment, the pacing electrode configuration to be used can also be selected. For example, the clinician can elect to use LV band to RV ring pacing (1967 in FIG. 19) or LV inter-band pacing (2067 in FIG. 20) as can be used when using segmented electrodes are employed. In one embodiment of the invention, the ET system is contained within the pacemaker can (e.g. internally generated orthogonal fields) and the auto-optimization cycle can be operated continuously.

An additional feature of the software is the option to select various left ventricular performance indices to be auto-optimized, including, but not limited to, stroke volume, cardiac output, ejection fraction, dP/dtₘₐₓ, strain rateₘₐₓ, peak systolic mitral annular velocity, end-systolic volume, end-diastolic volume, and QRS length. The clinician can obtain baseline information on left ventricular performance, then select a button to initiate an optimization routine. The software can automatically optimize for a particular left ventricular performance parameter of interest.

In addition to a three dimensional display of cardiac leads and electrodes, in certain embodiments the display can be in the form of a bar graph (FIG. 20). The display can include a comparison of selected cardiac parameters of interest at baseline (2056), and during pacing with different electrodes (2057). Each parameter can be depicted by a bar in a unique color; e.g. in FIG. 20 the parameters chosen for display are synchrony (blue), contractility (orange), and ejection fraction (green). The y-axis of the bar graph can be color-coded as well, with a color key similar to that described for FIG. 19 (see 2058; for example, green for a higher, or good, index or measurement; red for a lower, or suboptimal, index or measurement).

The display shown in FIG. 20 can also indicate the presence of phrenic nerve capture (undesirable stimulation of the diaphragm), which is clearly indicated on the ET voltage data as a sharp spike. The display can also include depiction of the ‘phrenic threshold’ (the stimulation level above which stimulation of the diaphragm occurs) (2059) and the ‘pacing threshold’ (the stimulation level which must be reached in order to achieve cardiac pacing) (2060). The system and display can include autodetection of phrenic nerve capture (2068), and can automatically adjust the stimulation strengths to increase thresholds to avoid phrenic nerve capture as part of the auto-optimize feature. If the phrenic nerve capture threshold is too low, the auto-optimize feature can include a test cycle of “intra-band” pacing, in which combinations of electrodes within a single location (e.g. a segmented electrode, 2061 in FIG. 20) that has been chosen for pacing can be tested to find the optimal combination that results in a high phrenic nerve capture threshold. Embodiments of such protocols are further described in PCT application serial no. PCT/US2006/012236 titled “Automated Optimization of Multi-electrode Pacing for Cardiac Resynchronization,” filed on Mar. 31, 2006; the disclosure of which is herein incorporated by reference.

Similarly, the display can also indicate optimal cardiac pacing thresholds. The auto-optimize feature can include a test cycle of “intra-band” pacing, in which combinations of pacing electrodes within a single location (e.g. a segmented electrode) that has been chosen for pacing can be tested, to locate the electrode facing the heart (2068, 2061). The magnitude of the EMG signals (the internal EKG, or local electric depolarization) can be tested to find the optimal location that results in a low cardiac pacing threshold. Embodiments of such protocols are further described in PCT application serial no. PCT/US2006/012236 titled “Automated Optimization of Multi-electrode Pacing for Cardiac Resynchronization,” filed on Mar. 31, 2006; the disclosure of which is herein incorporated by reference.

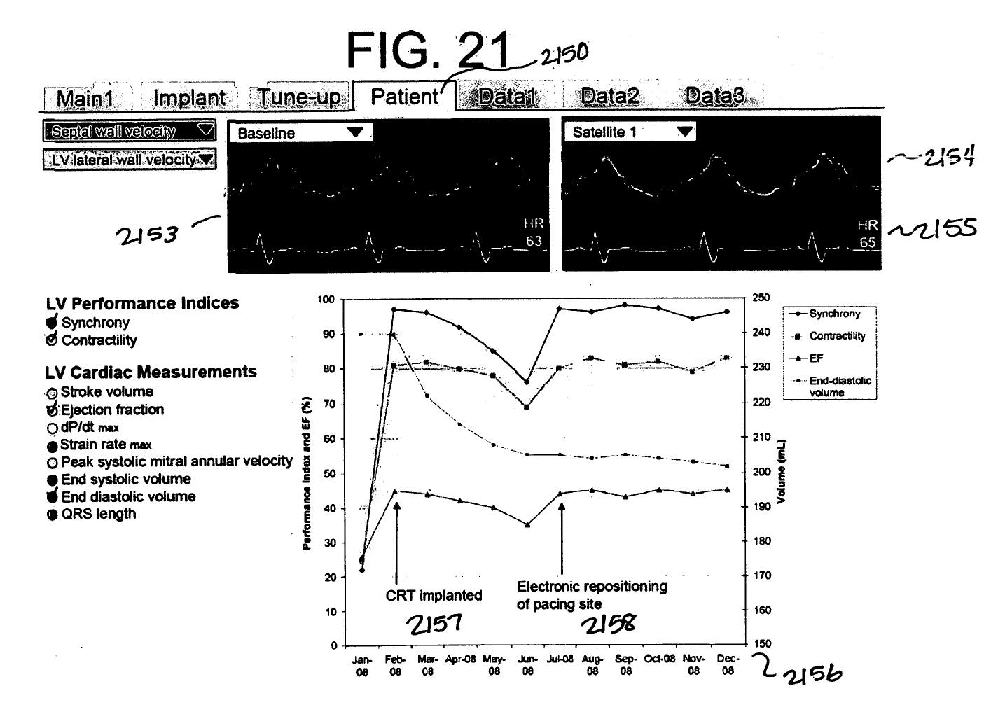

Another display option useful for long-term tracking of cardiac performance includes a method of displaying cardiac performance parameters of interest over time, e.g. over a period of months, or a year or more, in a two-dimensional graph format (FIG. 21). In one embodiment, the desired display can be chosen from a menu bar at the top of the display screen depending on the task for be performed; in this example “Patient” can be chosen when long-term tracking of a parameters for an individual patient is desired (2150 in FIG. 21) One or more LV performance indices and/or LV cardiac measurements can be chosen from the display and plotted as a function of time. In this way a clinician can compare cardiac performance parameters at baseline, and after some time period of CRT therapy, with the goal of allowing a clinician to observe the effect of CRT therapy, pharmacologic therapy, etc. on heart failure patients.

An example of long-term data tracking is shown in FIG. 21. One or more cardiac performance parameters (e.g. left ventricular end-diastolic volume) can be followed to observe effects of a clinical intervention or a change in drug regimen on cardiac performance (e.g. change in contractility). Each parameter can be depicted on the graph by a unique color and a unique shape marking data points; e.g. in FIG. 21 (2156) the parameters chosen for long-term tracking are synchrony (shown by a blue line With diamonds), contractility (shown by a yellow line with squares), end-diastolic volume (shown as a brown line with smaller squares), and ejection fraction (shown as green line with triangles). In certain embodiments, LV performance indices displayed can include, but are not limited to, synchrony and contractility. LV cardiac measurements displayed can include, but are not limited to, stroke volume, cardiac output, ejection fraction, dP/dtmax, strain ratemax, peak systolic mitral annular velocity, end-systolic volume, end-diastolic volume, and QRS length. In certain embodiments, the graph can also include markers indicating a clinical intervention (e.g. “CRT Implanted”, 2157; or “Electronic repositioning of pacing site”, 2158) or a change in drug regimen (e.g. an increased dose of a drug, such as a diuretic).

Where desired, the display can also include one or more additional plots and features, such as those described previously for FIGS. 19 and 20. In some embodiments this can include one or more additional plots of parameters of cardiac function, e.g. a graph of the degree of cardiac synchrony obtained at baseline (2153), and when using a particular pacing electrode (2154). The display can also include one or more additional plots derived from non-ET obtained data, such as a simultaneously obtained electrocardiogram (EKG) (2155).

Another method useful for evaluating the effectiveness of CRT is by displaying a two-dimensional ET velocity plot of velocity as determined by ET as a function of time. For example, the plot can show velocity of an electrode(s) on the left or posterior free wall of the heart, (e.g. linear velocity in the direction of the maximum motion as measured by ET, or linear velocity as measured by ET) at baseline, after pacing, and under different pacing conditions. The data can be from a single cardiac cycle, or from data averaged over several cardiac cycles, e.g. one cardiac cycle, two cardiac cycles, or three or more cardiac cycles. Tissue Doppler Imaging data, e.g., of the mitral annulus, can be acquired at a separate time. The ET velocity plot can be displayed in such a way that it substantially approximates a Tissue Doppler Imaging plot, e.g. of the mitral annulus, a display familiar to clinicians. The ET velocity data can be displayed either alone or along with the data obtained by TDI. FIG. 16 shows plots of data obtained from four different patients, demonstrating the correlation between ET data and TDI data. The white tracings in the plots show TDI velocity obtained from an echocardiogram at the mitral annulus, and the blue tracings show velocity in the direction of maximum motion of a left ventricular free wall electrode, derived from ET data. The display can also include a simultaneously obtained EKG. The display can include cardiac cycle event markers to identify events in the cardiac cycle (e.g. isovolumetric contraction, S-wave, E-wave, A-wave) in the EKG, the TDI data, and/or the ET velocity plot. For example, in FIG. 16 the timing and location of the S wave, and E wave, and the A wave are indicated on the ET velocity plot, as is an arrow identifying the length of one cardiac cycle.

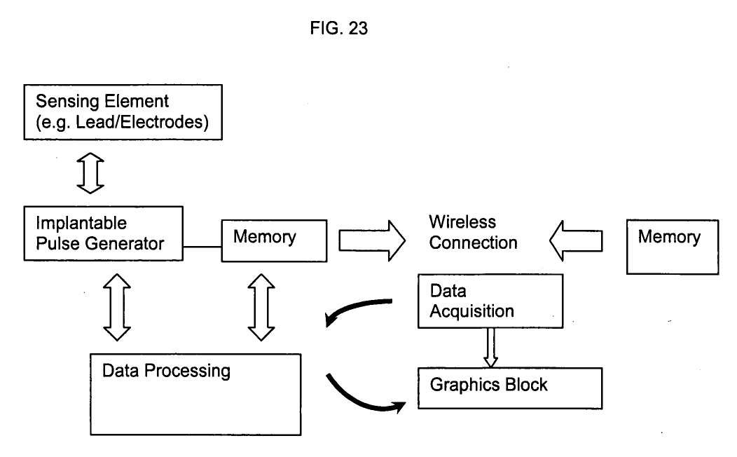

FIG. 23 is a schematic block diagram showing an exemplary implementation of a system for data acquisition, data processing, and display, in accordance with an embodiment of the invention. The system employed can include a data processing block connected to the implantable pulse generator, which can have algorithms including but not limited to algorithms for sequential electrode pacing, intraband pacing, etc. The method can also include a data acquisition block that can continuously ‘read’ data from the electrodes connected with the implantable pulse generator, and continuously recalculate position of the electrodes. The data processing and data acquisition blocks can both communicate with the graphics block, which can process the data obtained from the electrodes to generate multiple two-dimensional and three-dimensional displays as described above. The graphics block in certain embodiments can also include additional data in the display, e.g. fluoroscopy images as in the embodiment for lead implantation and placement described above. The method of the subject invention also includes one or more memory blocks, e.g. associated with the implantable pulse generator, either directly and/or by a wireless connection.

Applications

The electric field tomography methods of evaluating tissue location movement find use in a variety of different applications. As indicated above, an important application of the subject invention is for use in cardiac resynchronization, or CRT, also termed biventricular pacing. As is known in the art, CRT remedies the delayed left ventricular mechanics of heart failure patients. In a desynchronized heart, the interventricular septum will often contract ahead of portions of the free wall of the left ventricle. In such a situation, where the time course of ventricular contraction is prolonged, the aggregate amount of work performed by the left ventricle against the intraventricular pressure is substantial. However, the actual work delivered on the body in the form of stroke volume and effective cardiac output is lower than would otherwise be expected. Using the subject tomography approach, the electromechanical delay of the left lateral ventricle can be evaluated and the resultant data employed in CRT, e.g., using the approaches reviewed above and/or known in the art and reviewed at Col. 22, lines 5 to Col. 24, lines 34 of U.S. Pat. No. 6,795,732, the disclosure of which is herein incorporated by reference.

In a fully implantable system the location of the pacing electrodes on multi electrode leads and pacing timing parameters may be continuously optimized by the pacemaker. The pacemaker frequently determines the location and parameters which minimizes intraventricular dyssynchrony, interventricular dyssynchrony, or electromechanical delay of the left ventricle lateral wall in order to optimize CRT. This cardiac wall motion sensing system can also be used during the placement procedure of the cardiac leads in order to optimize CRT. An external controller could be connected to the cardiac leads and a skin patch electrode during placement of the leads. The skin patch acts as the reference electrode until the pacemaker is connected to the leads. In this scenario, for example, the optimal left ventricle cardiac vein location for CRT is determined by acutely measuring intraventricular dyssynchrony.

The subject methods and devices can be used to adjust a resynchronization pacemaker either acutely in an open loop fashion or on a nearly continuous basis in a closed loop fashion.

In certain embodiments, the systems and methods are employed to measure coupling between other electrode locations. The placement and selection of electrode pairs will determine the physical phenomenon that is measured. For instance the voltage coupling between an electrode in the right ventricle and an electrode in the right atrium provides an indication of the timing of the tricuspid valve closing and opening. In certain embodiments, a multiplicity of electrodes on a single lead. For instance a LV pacing lead might have electrodes in addition to the conventional pacing electrodes that extend from the vena cava, through the coronary sinus, and into a cardiac vein on the LV freewall. By selecting different pairs of these electrodes, different aspects of the heart's motion may be measured, as desired.

The subject methods and devices can also be employed in ischemia detection. It is well understood that in the event of acute ischemic events one of the first indications of such ischemia is akinesis, i.e., decreased wall motion of the ischemic tissue as the muscle becomes stiffened. As such, the present methods and devices provide a very sensitive indicator of an ischemic process, by ratiometrically comparing the local wall motion to a global parameter such as pressure. One can derive important information about unmonitored wall segments and their potential ischemia. For example, if an unmonitored section became ischemic, the monitored segment would have to work harder and have relatively greater motion in order to maintain systemic pressure and therefore ratio metric analysis would reveal that fact.

The subject methods and devices also find use in arrhythmia detection applications. Current arrhythmia detection circuits rely on electrical activity within the heart. Such algorithms are therefore susceptible to confusing electrical noise for an arrhythmia. There is also the potential for misidentifying or mischaracterizing arrhythmia based on electrical events when mechanical analysis would reveal a different underlying physiologic process. Accordingly,

Additional applications in which the subject invention finds use include, but are not limited to: the detection of electromechanical dissociation during pacing or arrhythmias, differentiation of hemodynamically significant and insignificant ventricular tachycardias, monitoring of cardiac output, mechanical confirmation of capture or loss of capture for autocapture algorithms, optimization of multi-site pacing for heart failure, rate responsive pacing based on myocardial contractility, detection of syncope, detection or classification of atrial and ventricular tachyarrhythmias, automatic adjustment of sense amplifier sensitivity based on detection of mechanical events, determination of pacemaker mode switching, determining the need for fast and aggressive versus slower and less aggressive anti-tachyarrhythmia therapies, or determining the need to compensate for a weakly beating heart after therapy delivery (where these representative applications are reviewed in greater detail in U.S. Pat. No. 6,795,732, the disclosure of which is herein incorporated by reference), and the like.

In certain embodiments, the subject invention is employed to overcome barriers to advances in the pharmacologic management of CHF, which advances are slowed by the inability to physiologically stratify patients and individually evaluate response to variations in therapy. It is widely accepted that optimal medical therapy for CHF involves the simultaneous administration of several pharmacologic agents. Progress in adding new agents or adjusting the relative doses of existing agents is slowed by the need to rely solely on time-consuming and expensive long-term morbidity and mortality trials. In addition, the presumed homogeneity of clinical trial patient populations may often be erroneous since patients in similar symptomatic categories are often assumed to be physiologically similar. It is desirable to provide implantable systems designed to capture important cardiac performance and patient compliance data so that acute effects of medication regimen variation may be accurately quantified. This may lead to surrogate endpoints valuable in designing improved drug treatment regimens for eventual testing in longer-term randomized morbidity and mortality studies. In addition, quantitative hemodynamic analysis may permit better segregation of drug responders from non-responders thereby allowing therapies with promising effects to be detected, appropriately evaluated and eventually approved for marketing. The present invention allows for the above. In certain embodiments, the present invention is used in conjunction with the a system as described in PCT Application Serial No. PCT/US2006/016370 titled “Pharma-Informatics System” and filed on Apr. 28, 2006; the disclosure of which is herein incorporated by reference.

In certain embodiments, electrodes (e.g. a multi-electrode lead) can be placed in the heart which are connected to the receiver, which can be employed to measure cardiac parameters of interest, e.g., blood temperature, heart rate, blood pressure, movement data, including synchrony data, as well as pharmaceutical therapy compliance. The obtained data is stored in the receiver. Embodiments of this configuration may be employed as an early heart failure diagnostic tool. This configuration may be put into a subject in the early stages of heart failure, with the goal of monitoring them closely and keeping them stable with optimized therapeutic management. Ultimately, when stimulation therapy is required, the receiver may be replaced with an implantable pulse generator, which may then employ the stimulating electrodes to provide appropriate pacing therapy to the subject.

Non-cardiac applications will be readily apparent to the skilled artisan, such as, by example, measuring the congestion in the lungs, determining how much fluid is in the brain, assessing distention of the urinary bladder. Other applications also include assessing variable characteristics of many organs of the body such as the stomach. In that case, after someone has taken a meal, the present invention allows measurement of the stomach to determine that this has occurred. Because of the inherently numeric nature of the data from the present invention, these patients can be automatically stimulated to stop eating, in the case of overeating, or encouraged to eat, in the case of anorexia. The present inventive system can also be employed to measure the fluid fill of a patient's legs to assess edema, or other various clinical applications.

Applications Using RV Lead

In certain embodiments, aspects of the invention above are employed in methods where cardiac function of a subject is evaluated by first applying an electric field across a right ventrical target location or site of said subject and then obtaining a signal from an electrode stably associated with said right ventrical target location. The resultant signal is then employed to evaluate cardiac function of the subject. As such, these embodiments include using electric tomography for determining general cardiac performance by tracking of just an RV lead, which can be a commercial RV lead. One can use an RV lead that is already implanted.

In certain embodiments, external skin patches, e.g., as described above, are employed to generate a 3D electric field externally, e.g., using patches on the chest, each on the right and left side for x, and on the chest on the back for the z and the leg and the neck for y, x-y-z field. Within that field the motion of the electrodes is tracked as they are moving around in these various electrical field gradients that have been generated, e.g., at 80 kHz to 100 kHz for each field. The resultant signals from any of the electrodes implanted in the heart and from their position data are employed to derive velocity, and even acceleration where desired. By looking at the velocity of these electrodes or the maximum peak velocity during systole, information about the global cardiac performance, e.g., how well the heart contracts and is synchronous, is obtained.

In certain embodiments, by just looking at an RV tip electrode, and by tracking the motion, looking at the maximum velocity of that electrode, a high correlation to LV dp/dt max, e.g., as obtained using a convention pressure sensor based protocol, is obtained. As such, embodiments of the methods include employing a right side of the heart sensing element to obtain information about a lefts side of the hear parameter of interest. Such an approach is desirable because placing devices into the left ventricle an increase the risk of thrombosis or such. The right side of the heart is very accessible to place RA leads and RV leads. Aspects of the invention include obtaining left ventricle information from a right ventricle device.

System components that can be employed in such methods include an RV lead, a pacemaker can that is used to deliver the stimulation to the heart, and in between these two devices an add-on module you would plug the lead into, e.g., as described above. This add-on module can plug into the pacemaker can. The module is essentially the brains for the electric tomography. It allows one to measure signals, voltage changes from the electrodes, and then via a wireless communication protocol, e.g., telemetry, sends out a signal to an external receiver which receives all the obtained data. As discussed before, the receiver can take the data and drop it into a programmer which has any convenient graphic user interface, computes all your data and shows traces of velocity for the electrode and picks out various parameters (for example maximum velocity). In one embodiment, a commercial pacemaker from any company (e.g., an off-the-shelf pacemaker), which can be a CRT or ICD pacemaker; whether it is doing LV, RV or if it is just doing RV side pacing, can be employed. Any off-the-shelf pacemaker can connect to the module, and on the other side any convenient RV lead can be connected. This system serves as a motion tracking system, and information about global contraction of the heart can be obtained with the system.

This system is in certain embodiments, an independent module. In other embodiments, the system can be an integrated feature to a pacemaker can or analogous implantable device. The system can be designed in a way that it would be invisible to the can). In certain embodiments, the pacemaker can does not even see the module, just wires going straight to the electrode. The pacemaker can paces right through the module. It can be coupled via capacitance, so it would be truly invisible to the pacemaker can. This module can be integrated into the can. The lead would plug into the IS-1 connecter into the module, and the module may have some pigtails which can plug into the can.

Multiple connections on the module can be provided, which would allow plug-in of any commercial RV lead, RA lead, and LV leads. In certain embodiments, such leads are hardwired leads, and ports would be provided on the module that can plug-in with whichever lead one wishes to use. In certain embodiments, auniversal module is employed. Where desired, the system may be compatible with a multi-electrode system that would have its own port for the multi-electrode lead that has chips that need to be talked to and programmed.

In certain embodiments, the system is used in a patient that is having new hardware put in for the first time. While putting leads into the heart the module is put into place. Also, any patient that comes in that needs their generator or battery changed can have this module added. They come in and open up the pocket, pull out the old pacemaker and snap the module into place along with a new pacemaker. Now this patient is a suitable for electric tomography and heart tracking. The present invention provides health care personnel, e.g., a heart failure specialist, with a way of tracking a real measure of heart performance in addition to some other more invasive ways he has right now. Tracking can be done periodically and a GUI that talks to that longitudinal display of how heart performance has changed over a year, 2 years, or 3 years with different drug regiments and such, can be done. How you can track the drugs.

The information may be extracted from the module via telemetry. For example the patient comes in for a follow-up in the office, they put a number of electric patches on the patient, and during a stress test (for example the patient is walking on the treadmill) one gets realistic information as to how the heart is really performing, not when the patient is just laying down. The telemetry would send out its signal to an antenna on the programmer which is sitting in the same room (sending it wirelessly). This module opens up a tremendous amount of patient population, not just CRT, any patient that has a pacemaker or is about to get a right-sided pacemaker, ICD, CRT-D or CRT-P. And of course it works with any lead or pacemaker that is currently available. Two designs for the RV tips are: (1) screw in, which basically attaches and you get direct contact with the tissue and such, and (2) it ties, it has little anchors protruding at the tip of the lead.

The obtained data can be stored onboard and then downloaded when the patient gets to the doctor's office, or with this day of home telemetry, i.e. medical telemetry, the data can be collected from home. In certain embodiments the module is not kept continuously running because it takes up power. In these embodiments once a day (or some other desirable interval) there is a quick measurement and it turns itself off. Then when the patient goes to bed, the device sends a signal over to their bedside telemetry system, which then sends the signal over to the doctor's office. Certainly a combination of telemedicine could be incorporated into this if you are using internal fields.

If the module was integrated then that would be the straight forward way to use the battery and the coil that the can uses for communicating. If it is outside other power sources can be employed, or recharge the battery in the module type or barnacle which is more for a multi-electrode lead. If the module is only running intermittently, a smaller batter is all that is required and the module just needs to last as long as the pacemaker can does, which is about 10 yrs. The module can go to a sleep function or just listens for a ping for when it needs to turn on, and takes some measurements and sends out data.

In certain embodiments, the system is provided in the form of a system that has a plastic header; in it is a molded standard IS-1 or DF connection that looks identical to the pacemaker. All the leads out from the heart are plugged into identical connections in the module, and tightened down the same way as the pacemaker. The connection side to the module, then on the other side of that header pigtail leads are present, proximal end of a lead sticking out from the module; so that now the pacemaker can see what it usually sees. The module has identical IS-1 connectors, silicone, coils, and then below the plastic header that is present in a titanium hermetically sealed laser welded can, with ceramic feed-thru to bring in the signal into the can. Inside the can are the electronics, battery for power and the coil, either inside the can or maybe integrated into the header to send out the RF telemetry signal.

Computer Readable Storage Media

One or more aspects of the subject invention may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

More specifically, computer readable medium may include stored programming embodying an algorithm for carrying out the subject methods. Accordingly, such a stored algorithm is configured to, or is otherwise capable of, practicing the subject methods, e.g., by operating an implantable medical device to perform the subject methods. The subject algorithm and associated processor may also be capable of implementing the appropriate adjustment(s).

Of particular interest in certain embodiments are systems loaded with such computer readable mediums such that the systems are configured to practice the subject methods.

Kits

As summarized above, also provided are kits for use in practicing the subject methods. The kits at least include a computer readable medium, as described above. The computer readable medium may be a component of other devices or systems, or components thereof, in the kit, such as an adaptor module, a pacemaker, etc. The kits and systems may also include a number of optional components that find use with the subject energy sources, including but not limited to, implantation devices, etc.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

As is evident from the above results and discussion, the subject invention provides numerous advantages. Advantages of various embodiments of the subject invention include, but are not limited to: low power consumption; real time discrimination of multiple lines of position possible (one or more); and noise tolerance, since the indicators are relative and mainly of interest in the time domain. A further advantage of this approach is that there is no need for additional catheters or electrodes for determining position. Rather the existing electrodes already used for pacing and defibrillation can be used to inject AC impulses at one or more frequencies designed not to interfere with the body or pacing apparatus. As such, the subject invention represents a significant contribution to the art.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for obtaining a parameter in a subject, said method comprising:
(a) generating an electric field so that a tissue site is present in said electric field; and
(b) employing a signal from a first sense electrode stably associated with said tissue site to obtain said parameter.

2. The method according to claim 1, wherein said method comprises generating a single electric field.

3. The method according to claim 2, wherein said single electric field is oriented in a direction of motion of interest.

4. The method according to claim 3, wherein said single electric field is reoriented at least once over a given period of time.

5. The method according to claim 1, wherein said method comprises generating two or more electric fields.

6. The method according to claim 5, wherein said method comprises generating three electric fields.

7. The method according to claim 6, wherein said method comprises generating three substantially orthogonal electric fields.

8. The method according to claim 1, wherein said method comprises generating more than three electric fields.

9. The method according to claim 8, wherein said method comprises generating six electric fields.

10. The method according to claim 1, wherein said signal is a voltage.

11. The method according to claim 1, wherein said method further comprises employing a signal from a second sense electrode stably associated with a second tissue site.

12. The method according to claim 1, wherein said parameter is a cardiac parameter.

13. The method according to claim 1, wherein said parameter is selected from the group consisting of:
ejection fraction, cardiac output, stroke volume, LV volume, LV diameter, LV end diastolic volume, LV end diastolic diameter, LV end systolic volume, LV end systolic diameter, contractility, dP/dt, dP/dtₘₐₓ, LV twist index, mitral regurgitation, myocardial strain, myocardial strain rate, myocardial strain rateₘₐₓ, myocardial position, cardiac wall motion, interventricular synchrony, intraventricular synchrony, septal lateral wall motion delay, septal posterior wall motion delay (SPWMD), interventricular mechanical delay (IVMD), mitral annular position, inter-electrode distances, isovolumetric relaxation time (IVRT), deceleration time (DT), atrial filling period (A_{dur}, at annulus), time from mitral valve opening to E velocity, beat-to-beat variability, valve timing, QRS duration, myocardial velocity, myocardial acceleration, systolic velocity, time to onset of systolic velocity, time to peak systolic velocity, time to peak post-systolic velocity, ET systolic measurements, Sₘ-ET (maximal velocity of a segment of myocardium), time to maximal systolic displacement (Td), e-wave velocity, a-wave velocity, mitral annular velocity, peak systolic mitral annular velocity, mitral annular acceleration, ET diastolic measurements, Eₐ-ET (maximal velocity of the mitral valve annulus during early diastolic filling), peak acceleration, time to peak acceleration, early diastolic filling velocity (E), filling velocity after atrial contraction (A), ratio of E/A, maximal acceleration, early diastolic deceleration slope, peak rapid filling rate, peak atrial filling rate, fractional filling rates, early diastolic myocardial tissue velocity (Eₘ), diastolic myocardial tissue velocity after atrial contraction (Aₘ), ratio of Eₘ/Aₘ, propagation velocity, rate of decline in LV pressure in early diastole (−dP/dt), left atrial pressure, ventricular pressure, end diastolic pressure, end systolic pressure, aortic pressure, valvular gradient, valvular regurgitation, blood flow, and mitral valve flow.

14. The method according to claim 1, wherein said parameter is selected from the group consisting of: transthoracic impedance, cardiac capture threshold, phrenic nerve capture threshold, temperature, respiratory rate, activity rate, hematocrit, heart sounds, sleep apnea determination.

15. The method according to claim 1, wherein said electric field is generated internally.

16. The method according to claim 1, wherein said electric field is generated externally.

17. The method according to claim 1, wherein said sense electrode is not present on a lead.

18. The method according to claim 1, wherein said sense electrode is present on carrier.

19. The method according to claim 18, wherein said carrier is a lead.

20. The method according to claim 18, wherein said carrier is a guidewire.

21. The method according to claim 18, wherein said carrier is a sheath.

22. The method according to claim 19, wherein said lead comprises a single sense electrode.

23. The method according to claim 19, wherein said lead is a multi-electrode lead.

24. The method according to claim 23, wherein said multi-electrode lead is a multiplex lead.

25. The method according to claim 23, wherein said multi-electrode lead comprises a segmented electrode.

26-30. (canceled)

31. A system for evaluating movement of a tissue location, said system comprising:
(a) an electric field generation element;
(b) a sense electrode configured to be stably associated with a cardiac tissue location; and
(c) a signal processing element configured to employ a signal obtained from said sense electrode to evaluate movement of tissue in a method according to claim 1.

32-41. (canceled)

42. A computer readable storage medium having a processing program stored thereon, wherein said processing program operates a processor to operate a system according to claim 31 to perform a method according to claim 1.

43. (canceled)

44. A method for evaluating movement of a first cardiac tissue at a site within a subject, said method comprising:
(a) generating an electric field so that said tissue is present in said electric field;
(b) monitoring voltage at a first sense electrode stably associated with said first cardiac tissue at said site to obtain data; and
(c) using said data to evaluate movement of said first cardiac tissue at said site within said subject.

45. The method according to claim 44, wherein said method comprises generating a single electric field.

46. The method according to claim 45, wherein said single electric field is oriented in direction of motion of interest.

47. The method according to claim 46, wherein said single electric field is reoriented at least once over a given period of time.

48. The method according to claim 44, wherein said method comprises generating two or more electric fields.

49. The method according to claim 48, wherein said method comprises generating three electric fields.

50. The method according to claim 49, wherein said method comprises generating three substantially orthogonal electric fields.

51. The method according to claim 44, wherein said method comprises generating more than three electric fields.

52. The method according to claim 51, wherein said method comprises generating six electric fields.

53-112. (canceled)
